SEQUENCE LISTING (62 sequences)

SEQ ID NO: 1 - protein, 18 aa, Artificial
  SHAAGtide
MLWRRKIGPQMTLSHAAG

SEQ ID NO: 2 - protein, 15 aa, Artificial
  SHAAGtide variant
RRKIGPQMTLSHAAG

SEQ ID NO: 3 - protein, 15 aa, Artificial
  SHAAGtide variant
MLWRRKIGPQMTLSH

SEQ ID NO: 4 - protein, 12 aa, Artificial
  SHAAGtide variant
IGPQMTLSHAAG

SEQ ID NO: 5 - protein, 12 aa, Artificial
  SHAAGtide variant
MLWRRKIGPQMT

SEQ ID NO: 6 - protein, 18 aa, Artificial
  SHAAGtide variant
MLWRRKIGPQMTLSHAAY

SEQ ID NO: 7 - protein, 16 aa, Artificial
  SHAAGtide variant
WRRKIGPQMTLSHAAG

SEQ ID NO: 8 - protein, 11 aa, Artificial
  SHAAGtide variant
MLWRRKIGPQM

SEQ ID NO: 9 - protein, 9 aa, Artificial
  SHAAGtide variant
WRRKIGPQM

SEQ ID NO: 10 - protein, 6 aa, Artificial
  SHAAGtide variant
WRRKIG

SEQ ID NO: 11 - protein, 14 aa, Artificial
  SHAAGtide variant
LWRRKIGPQMTLSH

SEQ ID NO: 12 - DNA, 54 bp, Artificial
  SHAAGtide polynucleotide
atgctctggaggagaaagattggtcctcagatgaccctttctcatgctgcagga

SEQ ID NO: 13 - DNA, 45 bp, Artificial
  Variant SHAAGtide polynucleotide
aggagaaagattggtcctcagatgaccctttctcatgctgcagga

SEQ ID NO: 14 - DNA, 45 bp, Artificial
  Variant SHAAGtide polynucleotide
atgctctggaggagaaagattggtcctcagatgaccctttctcat

SEQ ID NO: 15 - DNA, 36 bp, Artificial
  Variant SHAAGtide polynucleotide
attggtcctcagatgaccctttctcatgctgcagga

SEQ ID NO: 16 - DNA, 36 bp, Artificial
  Variant SHAAGtide polynucleotide
atgctctggaggagaaagattggtcctcagatgacc

SEQ ID NO: 17 - DNA, 54 bp, Artificial
  Variant SHAAGtide polynucleotide
atgctctggaggagaaagattggtcctcagatgaccctttctcatgctgcatat

SEQ ID NO: 18 - DNA, 48 bp, Artificial
  Variant SHAAGtide polynucleotide
tggaggagaaagattggtcctcagatgaccctttctcatgctgcagga

SEQ ID NO: 19 - DNA, 33 bp, Artificial
  Variant SHAAGtide polynucleotide
atgctctggaggagaaagattggtcctcagatg

SEQ ID NO: 20 - DNA, 27 bp, Artificial
  Variant SHAAGtide polynucleotide
tggaggagaaagattggtcctcagatg

SEQ ID NO: 21 - DNA, 18 bp, Artificial
  Variant SHAAGtide polynucleotide
tggaggagaaagattggt

SEQ ID NO: 22 - DNA, 42 bp, Artificial
  Variant SHAAGtide polynucleotide
ctctggaggagaaagattggtcctcagatgaccctttctcat

SEQ ID NO: 23 - DNA, 279 bp, Homo sapiens
atgctctggaggagaaagattggtcctcagatgaccctttctcatgctgcaggattccatgctactagtgctgactgctgcatctcctacaccccacgaagcatcccgtgttcactcctggagagttactttgaaacgaacagcgagtgctccaagccgggtgtcatcttcctcaccaagaaggggcgacgtttctgtgccaaccccagtgataagcaagttcaggtttgcatgagaatgctgaagctggacacacggatcaagaccaggaagaattga

SEQ ID NO: 24 - protein, 99 aa, Homo sapiens
RVTKDAETEFMMSKLPLENPVLLDRFHATSADCCISYTPRSIPCSLLESYFETNSECSKPGVIFLTKKGRRFCANPSDKQVQVCMRMLKLDTRIKTRKN

SEQ ID NO: 25 - protein, 75 aa, Homo sapiens
RFHATSADCCISYTPRSIPCSLLESYFETNSECSKPGVIFLTKKGRRFCANPSDKQVQVCMRMLKLDTRIKTRKN

SEQ ID NO: 26 - protein, 116 aa, Homo sapiens
RVTKDAETEFMMSKLPLENPVLLDMLWRRKIGPQMTLSHAAGFHATSADCCISYTPRSIPCSLLESYFETNSECSKPGVIFLTKKGRRFCANPSDKQVQVCMRMLKLDTRIKTRKN

SEQ ID NO: 27 - protein, 92 aa, Homo sapiens
MLWRRKIGPQMTLSHAAGFHATSADCCISYTPRSIPCSLLESYFETNSECSKPGVIFLTKKGRRFCANPSDKQVQVCMRMLKLDTRIKTRKN

SEQ ID NO: 28 - protein, 66 aa, Homo sapiens
SLAADTPTACCFSYTSRQIPQNFIADYFETSSQCSKPGVIFLTKRSRQVCADPSEEWVQKYVSDLE

SEQ ID NO: 29 - protein, 92 aa, Homo sapiens
QFINDAETELMMSKLPLENPVVLNSFHFAADCCTSYISQSIPCSLMKSYFETSSECSKPGVIFLTKKGRQVCAKPSGPGVQDCMKKLKPYSI

SEQ ID NO: 30 - protein, 68 aa, Homo sapiens
SFHFAADCCTSYISQSIPCSLMKSYFETSSECSKPGVIFLTKKGRQVCAKPSGPGVQDCMKKLKPYSI

SEQ ID NO: 31 - protein, 6 aa, Artificial
  W-peptide
HFYLPM

SEQ ID NO: 32 - protein, 6 aa, Artificial
  W-peptide
MFYLPM

SEQ ID NO: 33 - protein, 6 aa, Artificial
  W-peptide
  MOD_RES (6)..(6): D-Met
HFYLPM

SEQ ID NO: 34 - protein, 6 aa, Artificial
  W-peptide
  MOD_RES (6)..(6): D-Met
WKYMVM

SEQ ID NO: 35 - protein, 6 aa, Artificial
  W-peptide
  MOD_RES (6)..(6): D-Met
WKGMVM

SEQ ID NO: 36 - protein, 6 aa, Artificial
  W-peptide
  MOD_RES (6)..(6): D-Met
WKYMGM

SEQ ID NO: 37 - protein, 6 aa, Artificial
  W-peptide
WKYMVG

SEQ ID NO: 38 - protein, 6 aa, Artificial
  W-peptide
  MOD_RES (6)..(6): D-Met
WRYMVM

SEQ ID NO: 39 - protein, 6 aa, Artificial
  W-peptide
  MOD_RES (6)..(6): D-Met
WEYMVM

SEQ ID NO: 40 - protein, 6 aa, Artificial
  W-peptide
  MOD_RES (6)..(6): D-Met
WHYMVM

SEQ ID NO: 41 - protein, 6 aa, Artificial
  W-peptide
  MOD_RES (6)..(6): D-Met
WDYMVM

SEQ ID NO: 42 - protein, 6 aa, Artificial
  W-peptide
  MOD_RES (6)..(6): D-Met
WKHMVM

SEQ ID NO: 43 - protein, 6 aa, Artificial
  W-peptide
  MOD_RES (6)..(6): D-Met
WKEMVM

SEQ ID NO: 44 - protein, 6 aa, Artificial
  W-peptide
  MOD_RES (6)..(6): D-Met
WKWMVM

SEQ ID NO: 45 - protein, 6 aa, Artificial
  W-peptide
  MOD_RES (6)..(6): D-Met
WKRMVM

SEQ ID NO: 46 - protein, 6 aa, Artificial
  W-peptide
  MOD_RES (6)..(6): D-Met
WKDMVM

SEQ ID NO: 47 - protein, 6 aa, Artificial
  W-peptide
  MOD_RES (6)..(6): D-Met
WKFMVM

SEQ ID NO: 48 - protein, 6 aa, Artificial
  W-peptide
  MOD_RES (6)..(6): D-Met
WKYMYM

SEQ ID NO: 49 - protein, 6 aa, Artificial
  W-peptide
  MISC_FEATURE (5)..(5): Xaa is Phe or Trp
  MOD_RES (6)..(6): D-Met
WKYMXM

SEQ ID NO: 50 - protein, 6 aa, Artificial
  W-peptide
WKYMVE

SEQ ID NO: 51 - protein, 6 aa, Artificial
  W-peptide
WKYMVV

SEQ ID NO: 52 - protein, 6 aa, Artificial
  W-peptide
WKYMVR

SEQ ID NO: 53 - protein, 6 aa, Artificial
  W-peptide
WKYMVW

SEQ ID NO: 54 - protein, 5 aa, Artificial
  W-peptide
WKYMV

SEQ ID NO: 55 - protein, 5 aa, Artificial
  W-peptide
  MOD_RES (5)..(5): D-Met
KYMVM

SEQ ID NO: 56 - protein, 4 aa, Artificial
  W-peptide
KYMV

SEQ ID NO: 57 - protein, 4 aa, Artificial
  W-peptide
  MOD_RES (4)..(4): D-Met
YMVM

SEQ ID NO: 58 - protein, 3 aa, Artificial
  W-peptide
  MOD_RES (3)..(3): D-Met
MVM

SEQ ID NO: 59 - DNA, 20 bp, Artificial
  CpG-ODN 1668
tccatgacgttcctgatgct

SEQ ID NO: 60 - protein, 125 aa, Homo sapiens
QPDAVNAPLTCCYSFTSKMIPMSRLESYKRITSSRCPKEAVVFVTKLKREVCADPKKEWVQTYIKNLDRNQMRSEPTTLFKTASALRSSAPLNVKLTRKSEANASTTFSTTTSSTSVGVTSVTVN

SEQ ID NO: 61 - protein, 148 aa, Homo sapiens
MQVPVMLLGLLFTVAGWSIHVLAQPDAVNAPLTCCYSFTSKMIPMSRLESYKRITSSRCPKEAVVFVTKLKREVCADPKKEWVQTYIKNLDRNQMRSEPTTLFKTASALRSSAPLNVKLTRKSEANASTTFSTTTSSTSVGVTSVTVN

SEQ ID NO: 62 - protein, 77 aa, Homo sapiens
NLHLLRQKIEEQAAKYKHSVPKKCCYDGARVNFYETCEERVARVTIGPLCIRAFNECCTIANKIRKESPHKPVQLGR

(19) United States
(12) Patent Application Publication
(10) Pub. No.: US 2007/0087986 A1
(43) Pub. Date: Apr. 19, 2007

(54) Compositions and methods for enhancing immunity by chemoattractant adjuvants

(76) Inventors: Brett Premack, San Francisco, CA (US); Matthew J. Walters, Sunnyvale, CA (US); Thomas J. Schall, Palo Alto, CA (US)

Correspondence Address: Magdalena O. Cilella;Brinks Hofer Gilson & Lione, P.O. Box 10395, Chicago, IL, 60610, US

(21) Appl. No.: 11/496,267

(22) Filed: Jul. 31, 2006

Related U.S. Application Data
(63) Continuation-in-part of application No. 11/043,020, filed on Jan. 25, 2005.
(60) Provisional application No. 60/705,413, filed on Aug. 3, 2005.
(60) Provisional application No. 60/539,665, filed on Jan. 26, 2004.

(51) Int. Cl. A61K 48/00; A61K 38/19

(52) U.S. Cl. 514/44; 424/85.1

(57) ABSTRACT

The invention is directed to methods and compositions for enhancing immunity, including mucosal immunity. The compositions include an antigen or immunogen, a ligand for a Toll-like receptor and at least one chemoattractant.

### RELATED APPLICATIONS

The present patent document claims the benefit of the filing date under 35 U.S.C. §119(e) of Provisional U.S. Patent Application Ser. No. 60/705,413, filed Aug. 3, 2005 and is a continuation-in-part of application Ser. No. 11/043,020 filed Jan. 25, 2005, which claims the benefit of the filing date under 35 U.S.C. § 119(e) of Provisional U.S. Patent Application Ser. No. 60/593,665 filed Jan. 26, 2004. All of the foregoing applications are hereby incorporated by reference.

### FEDERALLY SPONSORED RESEARCH OR DEVELOPMENT

Sponsored by NIH-NIAID, Grant Number 1 U19 A1056690-01.

### BACKGROUND

1. Field of Invention

The present invention relates generally to methods and compositions for modulating immunity.

2. Background Information

There have been considerable efforts to identify substances that potentiate an immune response to an antigen, such as in a vaccine. Traditionally, effective vaccines contain two primary constituents, antigen and adjuvant and require efficient induction of antibody (Ab), type I interferons (IFN), cytokines/chemokines, cytotoxic T lymphocytes (CTL) and/or NK cells. However, often, vaccination, especially to peptides, is not immunogenic. Also, immunity, such as mucosal immunity for example, may not be achieved by standard routes of inoculation with an antigen, such as a peptide antigen.

To enhance immune response to an antigen or immunogen, adjuvants are often used. Adjuvants are known in the art as agents that enhance the efficacy and protective immune response of an immunogenic formulation, e.g., vaccines. Traditionally, the immunogenicity of a vaccine formulation has been improved by incorporating an adjuvant in the formulation. Immunological adjuvants were initially described by Ramon (1924, Ann. Inst. Pasteur, 38: 1) as “substances used in combination with a specific antigen that produced a more robust immune response than the antigen alone.” Adjuvants directly enhance the efficacy of the active ingredient, i.e., immunogenicity, in an immunogenic formulation.

An important aspect of the development of an adjuvant is the two-fold breadth potential for its application. Such breadth concerns both the underlying immune system and the target of the immune system's defense. An adjuvant can be useful in many mammalian species, if the adjuvant has the capacity to influence common attributes of the immune systems of those species. Further, if the targets of the immune system analogously have common attributes in how they are susceptible to immune responses, then an adjuvant stimulating an immune response should have broad applicability in defending against a variety of targets.

A wide variety of substances, both biological and synthetic, have been used as adjuvants.

One example of adjuvants currently approved by the U.S. Food and Drug administration are aluminum-based minerals (generically called Alum). Alum has a debatable safety record (see, e.g., Malakoff, 2000, Science, 288: 1323), and comparative studies show that it is a weak adjuvant for antibody induction to protein subunits and a poor adjuvant for cell-mediated immunity. Moreover, Alum adjuvants can induce IgE antibody response and have been associated with allergic reactions in some subjects (see, e.g., Gupta et al., 1998, Drug Deliv. Rev. 32: 155-72; Relyveld et al., 1998, Vaccine 16: 1016-23). Many experimental adjuvants have advanced to clinical trials since the development of Alum, and some have demonstrated high potency but have proven too toxic for therapeutic use in humans.

Toll-like receptors (TLRs) were also previously suggested to be adjuvant receptors that can sustain the molecular basis of adjuvant activity. Current consensus is that TLRs and their adapters introduce signals to preferentially induce IFN-α/β, chemokines, and pro-inflammatory cytokines, and mature mDC to augment antigen presentation. In other words, whenever TLR pathway is activated in mDC, NK and/or CTL activation is promoted.

TLRs are a family of receptors consisting of >10 protein members both in human and mice. Each TLR dimmer or a combination of TLRs can serve as receptor complex for the recognition of a specific microbial pattern molecule (Takeda K., et al., “Toll-Like Receptors” Annu Rev Immunol 2003;21:335-76; and Reis e Sousa C., “Toll-Like Receptors and Dendritic Cells: For Whom the Bug Tolls” Semin Immunol 2004;16:27-34). The recognition is then followed by TLR-specific signaling and corresponding cellular immune response (TABLE 1). Thus, the TLR family of proteins was found to serve as signaling receptors crucial for augmenting immune response. TLRs are present in myeloid dendritic cells (mDC), a representative cell population of antigen-presenting cells, and plasmacytoid dendritic cells (pDCs), formerly called type I interferon (IFN)-producing cells. Some TLRs are also present on T, B, NK cells and epithelial cells. mDCs are central to T/B cell activation. They facilitate production of antibodies through the induction of differentiation of B lymphocytes. T lymphocytes are differentiated by matured mDCs into T helper I (Th1), Th2, and CLT. mDCs catch up antigen, alter the function, and migrate to draining lymphnodes.

Exemplary TLRs, their adapters and exemplary TLR ligands are listed in TABLE 1 below adapted from Seya T., et al., “Role of Toll-Like Receptors in Adjuvant-Augmented Immune Therapies” eCAM 2006;3(1)31-38.

TABLE 1
Exemplary TLRs, their adapters and ligands.

| | Amino | | | Exemplary |
|---|---|---|---|---|
| huTLR | Acids | Adapters | DC Subset | Ligands |
| TLR1 | 786 | M-1/M-2 | M | Tracyl BLP |
| TLR2 | 784 | M-1/M-2 | M | PGN, BLP |
| TLR3 | 904 | T-1 (M-1) | M | dsRNA |
| TLR4 | 839 | M-1/M-2 | M | LPS, Taxol |
| TLR5 | 858 | M-1 | M | Flagellin |
| TLR6 | 796 | M-1/M-2 | M | Diacyl BLP |
| TLR7 | 1049 | M-1 | P | ssRNA |
| TLR8 | 1059 | M-1? | M | ssRNA |
| TLR9 | 1032 | M-1 | P | CpG DNA |
| M-1, MyD88; M-2, Mal/TIRAP; T-1, TICAM-1/TRIF; T-2, TICAM-2/TRAM. TLR1, TLR6, and TLR10 are members of the TLR2 subfamily and together with TLR2 recognize different sets of microbial pattern molecules and support activation of TLR2. | | | | |
| # Functional modes of each adapter were identified as ‘M’ and ‘T’ types. M, MyD88-dependent pathway; T, TICAM-1-dependent pathway. In DC subsets, ‘M’ is myeloid DCs while ‘P’ is plasmacytoid DCs. ‘Modes’ represent transcription factors activated by each TLR. | | | | |

TLR1, 2, 4, 5, and 6 are members of a TLR subfamily which recognizes microbial constituents that are absent in human cells. These TLR subfamily members reside on cell surface. Human mDCs express these TLR subfamily members. Human mDCs also express TLR3 and TLR8 while plasmacytoid dendritic cells (pDCs) express TLR7 and TLR9. These four TLRs preferably recognize microbe-specific modifications of nucleotide sequence and are localized in certain endosomes inside the cells (Seya T., et al, “Antibodies Against Human Toll-like Receptors (TLRs): TLR Distribution and Localization in Human Dendritic Cells” J Endotox Res 2005;11:369-74; and Iwasaki A., et al. “Toll-Like Receptor Control of the Adaptive Responses” Nat Immunol 2004;10:987-94). Human mDCs, however, do not express TLR9.

It is believed that TLR3, 7, 8, and 9 are proteins of TLR subfamily participating in the recognition of nucleic acid derivatives of viruses and bacteria.

Ligands for TLRs, including certain short bacterial immunostimulatory DNA sequences were proposed as adjuvants due to their ability to stimulate T helper-1 responses in animals vaccinated with genetic versions of antigens (Roman M. et al., 1997, Nature Med 3:849). These short DNA sequences are known as “CpG” sequences. The term “CpG” refers to dinucleotide sequences that are GC (Cytosine-Guanine)-rich DNA sequences. These CpG may be found within the promoter and first exon of approximately 50% of all genes in the human genome (Antequera & Bird, Proc. Natl. Acad. Sci. USA, 90: 11995-11999, 1993). CpG are ligands for the TLR9 receptor.

It is known in the art that CpG can stimulate the immune system, and can thereby be used to treat cancer, infectious diseases, allergy, asthma and other disorders, and to help protect against opportunistic infections following cancer chemotherapies. The cellular and humoral immune responses that result from CpG stimulation reflect the body's own natural defense system against invading pathogens and cancerous cells. CpG, while relatively rare in human DNA, are commonly found in the DNA of infectious organisms such as bacteria. The human immune system has apparently evolved to recognize CpG as an early warning sign of infection, and to initiate an immediate and powerful immune response against invading pathogens without causing adverse reactions frequently seen with other immune stimulatory agents. Thus, CpG, relying on this innate immune defense mechanism, can utilize a unique and natural pathway for immune therapy.

The effects of CpG on immune modulation were previously described extensively in U.S. Publication No. US20040198680 A1; U.S. Pat. Nos. 6,194,388; 6,207,646; 6,239,116 6,429,199; 6,214,806; 6,339,068; 6,406,705; and 6,218,371; and U.S. patent application Ser. No. 09/306,281 filed on May 6, 1999 (and related PCT/US99/09863). The entire contents of each of these patents and patent applications are hereby incorporated by reference.

However, although adjuvants were suggested for use in vaccine compositions, there is an unmet need for adjuvants that can effectively enhance immune response, and especially mucosal immune response, triggered by administered immunogen.

### SUMMARY

In one embodiment, the invention is an immunogenic composition including an antigen or immunogen, at least one chemoattractant, and a ligand for a Toll-like receptor (TLR). The ligand for the TLR is preferably a ligand for TLR9, such as a CpG oligonucleotide. The chemoattractant is preferably a chemokine or a W-tide. The chemokine may be IL-8, GCP-2, Gro α, Gro β, Gro γ, ENA-78, PBP, MIG, IP-10, I-TAC, SDF-1α (PBSF), BLC (BCA-1), MIP-1α, MIP-1β, RANTES, HCC-1, -2, -3, and -4, MCP-1, -2, -3, and -4, eotaxin-1, eotaxin-2, TARC, MDC, MIP-3α (LARC), MIP-3β (ELC), 6Ckine (LC), I-309, TECK, lymphotactin, fractalkine (neurotactin), TCA-4, Exodus-2, Exodus-3, or CKα-11. Preferably, the chemoattractant is a SHAAGtide polypeptide having at least 80% sequence identity to SEQ ID NO: 1. Preferably, the chemoattractant is a SHAAGtide polypeptide selected from the group consisting of SEQ ID NOS: 1, 2, 3, 4, 5, 6, 7, 8, 9, 10, and 11. The W-tide may be W-tide of SEQ ID NOS: 31, 32, 33, 34, 35, 36, 37, 38, 39, 40, 41, 42, 43, 44, 45, 46, 47, 48, 49, 50, 51, 52, 53, 54, 55, 56, 57, or 58. The immunogenic composition of this invention is preferably a vaccine composition. The antigen of the immunogenic composition of this invention may be a viral protein, polypeptide, or a fragment thereof; a cancer or tumor antigen; infectious disease agent; a bacterial agent; a parasite agent; or a fungal agent. Preferably, the immunogenic composition of this invention induces mucosal immunity is a subject. Preferably, the immunogenic composition of this invention enhances an antibody response in a subject. Preferably, the immunogenic composition of this invention is used for treatment of an infectious disease or cancer.

In another embodiment, the invention is a method of enhancing immunity. The method includes delivering to a subject an effective amount of a composition comprising an antigen, at least one chemoattractant, and a ligand for a Toll Like Receptor (TLR). Preferably, the method enhances mucosal immunity in a subject. The method may further comprise a step of measuring a level of immune response to the immunogenic composition, wherein the immune response is directed to the antigen or immunogen. The step of measuring the level of immune response to the antigen or immunogen may include determining humoral response. Alternatively, the step of measuring may include determining cell-mediated immune response.

In yet another embodiment, the invention is a method for identifying an adjuvant compound that enhances immunity to an antigen or immunogen. The method includes delivering an immunogenic composition comprising the antigen or immunogen, at least one chemoattractant and a ligand for a Toll-like receptor to a subject; and measuring a level of immune response to the immunogenic composition, wherein the immune response is directed to the antigen or immunogen.

In yet another embodiment, the invention is a method of inducing a mucosal immunity. The method includes applying to a mucosal membrane in a subject an effective amount of a composition comprising an antigen or immunogen, at least one chemoattractant, and a CpG. The mucosal membrane may be a vaginal membrane or a nasal membrane.

### BRIEF DESCRIPTION OF THE DRAWINGS

FIG. 1 shows the amino acid sequence alignment of the human CCL23/CKβ8 variants;

FIG. 2A-C shows a graph of serum levels of IgG specific for PA antigen induced by SHMGtide and CpG;

FIG. 3A-C shows a graph of levels of mucosal IgA induced by SHMGtide and CpG;

FIG. 4A-C shows a graph of serum levels of IgG specific for PA antigen induced by W-tide and CpG;

FIG. 5A-C shows a graph of levels of mucosal IgA induced by W-tide and CpG;

FIG. 6A-C shows a graph of serum levels of IgG specific for PA antigen induced by mJE and CpG;

FIG. 7A-C shows a graph of levels of mucosal IgA induced mJE and CpG;

FIG. 8A-C shows a graph of serum levels of IgG specific for PA antigen induced by mC5a and CpG;

FIG. 9A-C shows a graph of levels of mucosal IgA induced mC5a and CpG;

FIG. 10 shows a graph of anti-PA IgG1 levels in response to immunization with chemoattractants and CpG;

FIG. 11 shows a graph of anti-PA IgA levels in response to immunization with chemoattractants and CpG;

FIG. 12 shows a graph of anti-PA IgG1 levels in vaginal mucosa in response to immunization with chemoattractants and CpG;

FIG. 13 shows levels of serum IgG specific for anthrax protective antigen (PA ) in response to intra-nasal inoculation with PA antigen combined with Saline (negative control), SHMGtide, CpG, or a combination of SHMGtide and CpG;

FIG. 14 shows levels of serum IgG specific for PA antigen in response to intra-nasal inoculation with the PA antigen combined with Saline, Wtide, CpG, or a combination of Wtide and CpG;

FIG. 15 shows the titers of PA antigen-specific IgA recovered from the site of inoculation by nasal wash 27 days after intra-nasal inoculation; and

FIG. 16 shows levels of PA antigen-specific IgA recovered at a site distal to that of inoculation, e.g., from a vaginal wash 27 days after intra-nasal inoculation.

### DETAILED DESCRIPTION OF THE DRAWINGS AND THE PRESENTLY PREFERRED EMBODIMENTS

The invention is based on the unexpected discovery by the inventors that delivery of at least one chemoattractant together with an antigenic or immunogenic agent and a ligand for a Toll-like receptor (TLR) results in an enhanced immune response to the antigenic or immunogenic agent. Specifically, a mucosal immune response may be enhanced by addition of chemoattractant to a composition of an antigen or immunogen and a ligand for TLR for application to mucosal membranes.

I. Definitions

As used in this specification and the appended claims, the singular forms “a,” “an” and “the” include plural references unless the content clearly dictates otherwise.

Unless defined otherwise, all technical and scientific terms used herein have the meaning commonly understood by a person skilled in the art to which this invention belongs. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger et al. (eds.), Springer Verlag (1991); and Hale & Marham, THE HARPER COLLINS DICTIONARY OF BIOLOGY (1991).

The term “polypeptide” (e.g. SHAAGtide polypeptide, W-tide) is used interchangeably herein with the term “protein,” and refers to a polymer composed of amino acid residues linked by amide linkages, including synthetic, naturally-occurring and non-naturally occurring analogs thereof (amino acids and linkages). The terms do not refer to a specific length of the product. Thus, “peptides,” “oligopeptides,” and “proteins” are included within the definition of polypeptide. The terms include post-translational modifications of the polypeptide, for example, glycosylations, acetylations, phosphorylations and the like.

As used herein, references to specific proteins (e.g., FPRL1, SHMGtide, etc.) refers to a polypeptide having a native amino acid sequence, as well as variants and modified forms regardless of origin or mode of preparation. A protein that has a native amino acid sequence is a protein having the same amino acid sequence as obtained from nature. Such native sequence proteins can be isolated from nature or can be prepared using standard recombinant and/or synthetic methods. Native sequence proteins specifically encompass naturally occurring truncated or soluble forms, naturally occurring variant forms (e.g., alternatively spliced forms, such as CKβ8-1), naturally occurring allelic variants and forms including postranslational modifications. A native sequence protein includes proteins following post-translational modifications such as glycosylation of certain amino acid residues.

“Variants” refer to proteins that are functional equivalents to a native sequence protein that have similar amino acid sequences and retain, to some extent, one or more activities of the native protein. Variants also include fragments that retain activity. Representative activities of the chemokines, include, but are not limited to, ability to bind, activate, and/or modulate chemokine receptors and/or FPRL1 and modulate immune response. Exemplary activities of chemokine receptors include, but are not limited to, ability to bind cognate chemokine ligands, the ability to promote calcium mobilization, cell migration, and cell proliferation. Exemplary activities of FPRL1 include, but are not limited to, the ability to interact with SHAAGtides to regulate innate immune responses to a number of viral and bacterial pathogens. Exemplary activities of TLRs include ability to bind ligands, augment immune response and ability to recognize specific microbial pattern molecules.

Variants also include proteins that are substantially identical (see below) to a native sequence. Such variants include proteins having amino acid alterations such as deletions, insertions and/or substitutions. A “deletion” refers to the absence of one or more amino acid residues in the related protein. The term “insertion” refers to the addition of one or more amino acids in the related protein. A “substitution” refers to the replacement of one or more amino acid residues by another amino acid residue in the polypeptide. Typically, such alterations are conservative in nature such that the activity of the variant protein is substantially similar to a native sequence protein (see, e.g., Creighton (1984) Proteins, W. H. Freeman and Company). In the case of substitutions, the amino acid replacing another amino acid usually has similar structural and/or chemical properties. Insertions and deletions are typically in the range of 1 to 5 amino acids, although depending upon the location of the insertion, more amino acids can be inserted or removed. The variations can be made using methods known in the art such as site-directed mutagenesis (Carter, et al. (1986) Nucl. Acids Res. 13:4331; Zoller et al. (1987) Nucl. Acids Res. 10:6487), cassette mutagenesis (Wells et al. (1985) Gene 34:315), restriction selection mutagenesis (Wells, et al. (1986) Philos. Trans. R. Soc. London SerA 317:415), and PCR mutagenesis (Sambrook et al., Molecular Cloning: A Laboratory Manual, 3rd edition, Cold Spring Harbor Press, N.Y., (2001)).

Modified forms of a protein generally refer to proteins in which one or more amino acids of a native sequence have been altered to a non-naturally occurring amino acid residue. Such modifications can occur during or after translation and include, but are not limited to, phosphorylation, glycosylation, cross-linking, acylation and proteolytic cleavage.

The term “conservative substitution,” when describing a polypeptide, refers to a change in the amino acid composition of the polypeptide that does not substantially alter the activity of the polypeptide, i.e., substitution of amino acids with other amino acids having similar properties such that the substitutions of even critical amino acids does not substantially alter activity. Conservative substitution tables providing functionally similar amino acids are well known in the art. The following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W) (see also, Creighton, 1984, Proteins, W. H. Freeman and Company).

In addition to the above-defined conservative substitutions, other modification of amino acid residues can result in “conservatively modified variants.” For example, one may regard all charged amino acids as substitutions for each other whether they are positive or negative. In addition, conservatively modified variants can also result from individual substitutions, deletions or additions which alter, add or delete a single amino acid or a small percentage of amino acids, e.g., often less than 5%, in an encoded sequence. Further, a conservatively modified variant can be made from a recombinant polypeptide by substituting a codon for an amino acid employed by the native or wild-type gene with a different codon for the same amino acid.

The terms “nucleic acid,” “polynucleotide,” and “oligonucleotide” are used herein to include a polymeric form of nucleotides of any length, including, but not limited to, ribonucleotides or deoxyribonucleotides. There is no intended distinction in length between these terms. Further, these terms refer only to the primary structure of the molecule. Thus, in certain embodiments these terms can include triple-, double- and single-stranded DNA, as well as triple-, double- and single-stranded RNA. They also include modifications, such as by methylation and/or by capping, and unmodified forms of the polynucleotide. More particularly, the terms “nucleic acid,” “polynucleotide,” and “oligonucleotide,” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. Thus, in certain embodiments these terms include, for example, 3′-deoxy-2′, 5′-DNA, oligodeoxyribonucleotide N3′ P5′ phosphoramidates, 2′-O-alkyl-substituted RNA, double- and single-stranded DNA, as well as double- and single-stranded RNA, DNA:RNA hybrids, and hybrids between PNAs and DNA or RNA, and also include known types of modifications, for example, labels that are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine), those with intercalators (e.g., acridine, psoralen), those containing chelators (e.g., metals, radioactive metals, boron, oxidative metals), those containing alkylators, those with modified linkages (e.g., alpha anomeric nucleic acids), as well as unmodified forms of the polynucleotide or oligonucleotide. In particular, DNA is deoxyribonucleic acid.

A “heterologous sequence” or a “heterologous nucleic acid,” as used herein, is one that originates from a source foreign to the particular host cell, or, if from the same source, is modified from its original form. Thus, a heterologous gene in a prokaryotic host cell includes a gene that, although being endogenous to the particular host cell, has been modified. Modification of the heterologous sequence can occur, e.g., by treating the DNA with a restriction enzyme to generate a DNA fragment that is capable of being operably linked to the promoter. Techniques such as site-directed mutagenesis are also useful for modifying a heterologous nucleic acid.

The term “recombinant” when used with reference to a cell indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

A “recombinant expression cassette” or simply an “expression cassette” is a nucleic acid construct, generated recombinantly or synthetically, that has control elements that are capable of affecting expression of a structural gene that is operably linked to the control elements in hosts compatible with such sequences. Expression cassettes include at least promoters and optionally, transcription termination signals. Typically, the recombinant expression cassette includes at least a nucleic acid to be transcribed (e.g., a nucleic acid encoding UCP-2) and a promoter. Additional factors necessary or helpful in effecting expression can also be used as described herein. For example, transcription termination signals, enhancers, and other nucleic acid sequences that influence gene expression, can also be included in an expression cassette.

As used herein, the term “substantial sequence identity,” “sequence identity,” and “substantially identical” and other like phrases refers to two or more sequences or subsequences that have at least 60% or 70%, preferably 80% or 85%, most preferably 90%, 95%, 98%, or 99% nucleotide or amino acid residue identity, when compared and aligned for maximum correspondence, as measured using one of the following sequence comparison algorithms or by visual inspection. Two sequences (amino acid or nucleotide) can be compared over their full-length (e.g., the length of the shorter of the two, if they are of substantially different lengths) or over a subsequence such as at least 50, 100, 200, 500 or 1000 contiguous nucleotides or at least 10, 20, 30, 40, 50 or 100 contiguous amino acid residues.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., Current Protocols In Molecular Biology, Greene Publishing and Wiley-Interscience, New York (supplemented through 1999). Each of these references and algorithms is incorporated by reference herein in its entirety. When using any of the aforementioned algorithms, the default parameters for “Window” length. gap penalty, etc., are used.

One example of algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the first polypeptide (e.g., a polypeptide encoded by the first nucleic acid) is immunologically cross reactive with the second polypeptide (e.g., a polypeptide encoded by the second nucleic acid). Thus, a polypeptide is typically substantially identical to a second polypeptide, for example, where the two peptides differ only by conservative substitutions.

Another indication that two nucleic acid sequences are substantially identical is that the two molecules hybridize to each other under stringent conditions. Substantial identity exists when the segments will hybridize under stringent hybridization conditions to a strand, or its complement, typically using a sequence of at least about 50 contiguous nucleotides derived from the probe nucleotide sequences.

“Stringent hybridization conditions” refers to conditions in a range from about 5° C. to about 20° C. or 25° C. below the melting temperature (Tₘ) of the target sequence and a probe with exact or nearly exact complementarity to the target. As used herein, the melting temperature is the temperature at which a population of double-stranded nucleic acid molecules becomes half-dissociated into single strands. Methods for calculating the Tₘ of nucleic acids are well known in the art (see, e.g., Berger and Kimmel, 1987, Methods In Enzymology, Vol. 152: Guide To Molecular Cloning Techniques, San Diego: Academic Press, Inc. and Sambrook et al., Molecular Cloning: A Laboratory Manual, 3rd edition, Cold Spring Harbor Press, N.Y., (2001). As indicated by standard references, a simple estimate of the Tₘ value may be calculated by the equation: Tₘ=81.5+0.41 (% G+C), when a nucleic acid is in aqueous solution at 1 M NaCl (see e.g., Anderson and Young, “Quantitative Filter Hybridization” in Nucleic Acid Hybridization (1985)). Other references include more sophisticated computations which take structural as well as sequence characteristics into account for the calculation of Tₘ. The melting temperature of a hybrid (and thus the conditions for stringent hybridization) is affected by various factors such as the length and nature (DNA, RNA, base composition) of the probe and nature of the target (DNA, RNA, base composition, present in solution or immobilized, and the like), and the concentration of salts and other components (e.g., the presence or absence of formamide, dextran sulfate, polyethylene glycol). The effects of these factors are well known and are discussed in standard references in the art, see e.g., Sambrook et al., Molecular Cloning: A Laboratory Manual, 3rd edition, Cold Spring Harbor Press, N.Y., (2001), and Ausubel et al., Current Protocols In Molecular Biology, Greene Publishing and Wiley-Interscience, New York (supplemented through 1999). Typically, stringent hybridization conditions are salt concentrations less than about 1.0 M sodium ion, typically about 0.01 to 1.0 M sodium ion at pH 7.0 to 8.3, and temperatures at least about 30° C. for short probes (e.g., 10 to 50 nucleotides) and at least about 60° C. for long probes (e.g., greater than 50 nucleotides). As noted, stringent conditions may also be achieved with the addition of destabilizing agents such as formamide, in which case lower temperatures may be employed.

The terms “substantially pure” or “isolated,” when referring to proteins and polypeptides, denote those polypeptides that are separated from proteins or other contaminants with which they are naturally associated. A protein or polypeptide is considered substantially pure when that protein makes up greater than about 50% of the total protein content of the composition containing that protein, and typically, greater than about 60% of the total protein content. More typically, a substantially pure or isolated protein or polypeptide will make up at least 75%, more preferably, at least 90%, of the total protein. Preferably, the protein will make up greater than about 90%, and more preferably, greater than about 95% of the total protein in the composition.

The term “therapeutically effective amount” means the amount of the subject compound that will elicit the biological or medical response of a tissue, system, animal or human that is being sought by the researcher, veterinarian, medical doctor or other clinician.

By “enhancing an immune response” or “potentiating immune response” or “augmenting immune response” is meant any improvement in an immune response, including mucosal immune response, that has already been mounted by a mammal. By “inducing an immune response” is meant the initiation of an immune response, including mucosal immune response, against an antigen of interest in a mammal in which an immune response against the antigen of interest has not already been initiated. Both situations are included in this invention and words enhance, induce, potentiate, and augment will be used interchangeably. In both situations, the immune response can involve both the humoral and cell-mediated arms of the immune system. For further discussion of immune responses, see, e.g., Abbas et al. Cellular and Molecular Immunology, 3^{rd} Ed., W. B. Saunders Co., Philadelphia, Pa. (1997). Those of ordinary skill in the art recognize that there are variety of methods for assessing such enhancements, for example, humoral antibody measurements, cytokine measurements, assessing animal health or well-ness, clinical protection from disease, and measuring cell mediated changes in immunity.

As used herein a “vaccine composition” or “vaccine antigen composition” is any composition containing a molecule capable of stimulating an immune system response. This invention is particularly related to vaccines that stimulate an immune system response to an antigen, and more specifically to an antigen that is associated with a disease state of an animal. The compositions and methods of this invention particularly relate to vaccine compositions that comprise an antigen, a chemoattractant and a ligand for a Toll-like receptor. Vaccine compositions may contain other ingredients as known in the art to facilitate or benefit functionality. Vaccine dosage levels for a given application can be determined by well-known methods.

By “pharmaceutically acceptable” it is meant the carrier, diluent or excipient must be compatible with the other ingredients of the formulation and not deleterious to the recipient thereof.

A “control value” or simply “control” generally refers to a value (or range of values) against which an experimental or determined value is compared. Thus, in the case of a screening assay, the control value can be a value for a control reaction that is conducted under conditions that are identical those of a test assay, except that the control reaction is conducted in the absence of a candidate agent whereas the test assay is conducted in the presence of the candidate agent. The control value can also be a statistical value (e.g., an average or mean) determined for a plurality of control assays. The control assay(s) upon which the control value is determined can be conducted contemporaneously with the test or experimental assay or can be performed prior to the test assay. Thus, the control value may be based upon contemporaneous or historical controls.

A difference is typically considered to be “statistically significant” if in general terms an observed value differs from a control or background value by more than the level of experimental error. A difference can be considered “statistically significant” if the probability of the observed difference occurring by chance (the p-value) is less than some predetermined level. As used herein a “statistically significant difference” refers to a p-value that is <0.05, preferably <0.01 and most preferably <0.001.

The term “antibody” as used herein includes antibodies obtained from both polyclonal and monoclonal preparations, as well as the following: (i) hybrid (chimeric) antibody molecules (see, for example, Winter et al. (1991) Nature 349:293-299; and U.S. Pat. No. 4,816,567); (ii) F(ab′)2 and F(ab) fragments; (iii) Fv molecules (noncovalent heterodimers, see, for example, Inbar et al. (1972) Proc. Natl. Acad. Sci. USA 69:2659-2662; and Ehrlich et al. (1980) Biochem 19:4091-4096); (iv) single-chain Fv molecules (sFv) (see, for example, Huston et al. (1988) Proc. Natl. Acad. Sci. USA 85:5879-5883); (v) dimeric and trimeric antibody fragment constructs; (vi) humanized antibody molecules (see, for example, Riechmann et al. (1988) Nature 332:323-327; Verhoeyan et al. (1988) Science 239:1534-1536; and U.K. Patent Publication No. GB 2,276,169, published 21 Sep. 1994); (vii) Mini-antibodies or minibodies (i.e., sFv polypeptide chains that include oligomerization domains at their C-termini, separated from the sFv by a hinge region; see, e.g., Pack et al. (1992) Biochem 31:1579-1584; Cumber et al. (1992) J. Immunology 149B:120-126); and, (vii) any functional fragments obtained from such molecules, wherein such fragments retain specific-binding properties of the parent antibody molecule.

The phrases “specifically binds” when referring to a protein, “specifically immunologically cross reactive with,” or simply “specifically immunoreactive with” when referring to an antibody, refers to a binding reaction which is determinative of the presence of the protein in the presence of a heterogeneous population of proteins and other biologics. Thus, under designated conditions, a specified ligand binds preferentially to a particular protein and does not bind in a significant amount to other proteins present in the sample. A molecule or ligand (e.g., an antibody) that specifically binds to a protein has an association constant of at least 10³ M⁻¹ or 10⁴ M⁻¹, sometimes 10⁵ M⁻¹ or 10⁶ M⁻¹, in other instances 10⁶ M⁻¹ or 10⁷ M⁻¹, preferably 10⁸ M⁻¹ to 10⁹ M⁻¹, and more preferably, about 10¹⁰ M⁻¹ to 10¹¹ M⁻¹ or higher. A variety of immunoassay formats can be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

The term “detectably labeled” means that an agent (e.g., a probe) has been conjugated with a label that can be detected by physical, chemical, electromagnetic and other related analytical techniques. Examples of detectable labels that can be utilized include, but are not limited to, radioisotopes, fluorophores, chromophores, mass labels, electron dense particles, magnetic particles, spin labels, molecules that emit chemiluminescence, electrochemically active molecules, enzymes, cofactors, and enzyme substrates.

A “small organic molecule” or simply “small molecule” as used herein refers to a synthetic molecule that typically has a molecular weight of less than 1000 daltons, more typically 500 daltons or less. Such molecules can include, for example, sterols, amino acids, small nucleic acids, small peptides, aromatic compounds, heterocyclic compounds, benzodiazepines, oligomeric N-substituted glycines and oligocarbamates.

A “subject”, “individual” or “patient” as used herein in the context of therapeutic and prophylactic treatment methods typically refers to a mammal, including primates (e.g., humans, apes, chimpanzee, gorilla) and non-human primates (e.g., mouse, rat, rabbit), but most typically refers to a human.

II. Overview

The invention is based on the unexpected discovery that delivery of at least one chemoattractant with an antigenic or immunogenic agent and a known adjuvant, such as a ligand for a TLR (adjuvant) results in an enhanced immune response to the antigenic or immunogenic agent as compared to immune response resulting from delivery of antigenic or immunogenic agent alone or with a ligand for TLR, but without the chemoattractant. Specifically, an enhancement of mucosal immune response was observed when a chemoattractant was administered together with an antigen or immunogen and a ligand for TLR.

Although, TLR ligands, including CpG, which are ligands for TLR9, were previously used as adjuvant for vaccination as discussed above, Applicants have discovered that the adjuvant efficacy of ligands for TLRs, including CpG, in vaccinations, including nasal vaccinations, may be enhanced by including a chemoattractant. Specifically, when the ligand for TLR is a CpG, initiation of immunity at the mucosal surface upon addition of a chemoattractant to a CpG as an adjuvant is observed (Examples).

As such, this invention is directed to immunogenic compositions, including vaccine compositions, and methods for enhancing or inducing an immune response, including mucosal immune response, to an antigen or immunogen. This enhancement is by including a chemoattractant as an adjuvant to enhance the adjuvant activity of a ligand for TLR.

Specifically, the immunogenic composition of this invention includes an antigen or immunogen, a ligand for a TLR, and a chemoattractant to enhance the adjuvant activity of the ligand for TLR. The immunogenic composition of this invention may have an enhanced therapeutic efficacy, safety, and toxicity profile relative to other available formulations.

Exemplary and preferred chemoattractants for use in this invention are described in detail below.

Any suitable ligands for TLR, and especially those currently being used as adjuvants in immunogenic compositions may be used in this invention. Exemplary ligands for TLR were described in more detail above. Preferably, the TLR ligand is TLR9 ligand, such as CpG oligonucleotide, and more preferably, the CpG oligonucleotide is unmethylated.

The immunogenic composition of the invention is particularly effective in stimulating and/or up-regulating an antibody response to a level greater than that seen in conventional immunogenic compositions (such as vaccines with antigen only or with antigen in combination with a conventional adjuvant) and administration schedules. The immunogenic compositions of the invention are particularly advantageous for developing rapid and high levels of immunity against the antigenic or immunogenic agent, against which an immune response is desired. The immunogenic compositions of the invention may achieve a systemic immunity at a protective level with a low dose of the antigenic or immunogenic agent. In some embodiments, the immunogenic compositions of the invention may result in an enhanced immune response with a dose of the antigenic or immunogenic agent which is 60%, preferably 50%, more preferably 40% of the dose conventionally used for the antigenic or immunogenic agent in obtaining an effective immune response. In preferred embodiments, the immunogenic compositions of the invention comprise a dose of the antigenic or immunogenic agent which is lower than the conventional dose used in the art, e.g., the dose recommended in the Physician's Desk Reference, utilizing the conventional modes of delivery, e.g., intramuscular and subcutaneous and the conventional compositions, i.e., in the absence of chemoattractant adjuvant of the invention. Preferably, the immunogenic compositions of the invention result in a therapeutically or prophylactically effective immune response after a single dose.

Preferably, the immunogenic composition of the invention is a vaccine composition. The immunogenic composition may be administered in an effective-amount for inducing or potentiating an adjuvant response (i.e., immunity). Various modes of administration of vaccine compositions may be employed and are described in more detail below.

The invention also provides methods of treatment and prophylaxis which involve administering an immunogenic composition of the invention to a subject, preferably a mammal, and most preferably a human for treating, managing or ameliorating symptoms associated with a disease or disorder, especially an infectious disease or cancer.

The invention encompasses a method of enhancing immunity, including mucosal immunity. The method includes delivering to a subject an effective amount of a composition comprising an antigen, at least one chemoattractant, and a ligand for a TLR.

The invention encompasses a method for immunization and/or stimulating an immune response in a subject comprising delivery of a single dose of a composition of the invention to a subject, preferably a human. In some embodiments, the invention encompasses one or more booster immunizations.

The invention encompasses methods for determining the efficacy of immunogenic compositions of the invention using any standard method known in the art or described herein. Assays for determining the efficacy of the immunogenic compositions of the invention may be in vitro based assays or in vivo based assays, including animal based assays. In some embodiments, the invention encompasses detecting and/or quantitating a humoral immune response against the antigenic or immunogenic agent of a composition of the invention in a sample, e.g., serum, obtained from a subject who has been administered an immunogenic composition of the invention. Preferably, the humoral immune response of the immunogenic compositions of the invention is compared to a control sample obtained from the same subject, who has been administered a control formulation, e.g., a formulation which simply comprises of the antigenic or immunogenic agent or a formulation which simply comprises of the antigenic or immunogenic agent and a ligand for a Toll-like receptor.

In other embodiments, the invention encompasses methods for determining the efficacy of the compositions of the invention by measuring cell-mediated immune response. Methods for measuring cell-mediated immune response are known to one skilled in the art and encompassed within the invention. In some embodiments, a T cell immune response may be measured for quantitating the immune response in a subject, for example by measuring cytokine production using common methods known to one skilled in the art including but not limited to ELISA from tissue culture supernatants, flow cytometry based intracellular cytokine staining of cells ex vivo or after an in vitro culture period, and cytokine bead array flow cytometry based assay. In yet other embodiments, the invention encompasses measuring T cell specific responses using common methods known in the art, including but not limited to chromium based release assay, flow cytometry based tetramer or dimer staining assay using known CTL epitopes.

The invention further encompasses methods of identifying a chemoattractant compound that enhances an immune response to an immunogenic or antigenic agent. In one embodiment, a method of identifying a chemoattractant compound that enhances an immune response to an antigenic or immunogenic agent comprises: delivering an immunogenic composition to the subject, measuring a level of immune response, wherein the immunogenic composition comprises the immunogenic or antigenic agent, at least one ligand for a TLR, and a test chemoattractant compound and wherein the immune response is directed to the antigenic or immunogenic agent. The invention encompasses measuring a level of immune response by determining humoral and/or cell-mediated immune response using methods known to one skilled in the art and disclosed herein. Once a level of immune response is determined, it is compared to a standard level, wherein elevation of the measured level indicates that the test chemoattractant compound is an adjuvant.

In yet another embodiment, a method for identifying a compound that enhances immunogenicity of an immunogenic or antigenic agent comprises: (a) delivering an immunogenic composition to a first subject, wherein the immunogenic composition comprises the immunogenic or antigenic agent, a ligand for TLR, and a test chemoattractant compound; (b) measuring antibody response in a sample obtained from the first subject's serum; (c) delivering an immunogenic composition to a second subject, wherein the immunogenic composition comprises the immunogenic or antigenic agent, a ligand for TLR without the test chemoattractant compound, and wherein the first and the second subjects are same species; (d) measuring antibody response in a sample obtained from the second subject's serum; (e) determining whether the response obtained from the first subject is greater than the response obtained from the second subject. If the response in the sample obtained from the first subject is greater than the second subject, characterizing the test chemoattractant compound as an adjuvant that may be used in the compositions of the invention, and (f) demonstrating that the concentration of the chemoattractant that provides an adjuvant property is a concentration that produces acceptable immune response. Compounds identified by the screening methods of the invention can be used to elicit an enhanced immune response to an antigenic or immunogenic agent when co-administered with the antigenic or immunogenic agent and a ligand for a TLR into the subject. Specifically, these chemoattractants can be used in vaccine compositions.

The invention further encompasses kits comprising an immunogenic composition of the invention as described herein. In a specific embodiment, the invention provides a kit comprising, one or more containers filled with one or more of the components of the immunogenic compositions of the invention, e.g., an antigenic or immunogenic agent, a ligand for a TLR, and a chemoattractant. In another specific embodiment, the kit comprises two containers, one containing an antigenic or immunogenic agent, and the other containing the combination of the two adjuvants, namely a ligand for a TLR and a chemoattractant. Associated with such container(s) can be a notice in the form prescribed by a governmental agency regulating the manufacture, use or sale of pharmaceuticals or biological products, which notice reflects approval by the agency of manufacture, use or sale for human administration.

III. Chemoattractants and Chemoattractant Receptors

As previously mentioned, inventors discovered surprising adjuvant activity of chemoattractants in enhancing an immune response to an antigen or immunogen which is combined with a different adjuvant, such as a ligand for TLR.

Chemoattractants are well known in the art. The term “chemoattractants” refers to peptides or polypetides which induce cell migration, motility or activation by binding to chemoattractant receptors, typically seven transmembrane G-protein coupled receptors (GPCRs), as exemplified by the chemokine receptor family. Chemoattractants may also include viral homologs of mammalian chemokines, truncated or cleaved fragments of chemokines or small molecule mimetics which induce motility through chemoattractant receptors.

A “chemoattractant receptor” refers to a receptor that upon binding to a ligand induces cell migration; i.e., movement of cells towards an increasing concentration of a chemical. Some exemplary chemoattractant receptors and their ligands include those shown in TABLE 2.

TABLE 2
Exemplary human chemoattractant
receptors and exemplary ligands¹

| Receptor | Examples of ligands² |
|---|---|
| BLT1 | Leukotriene B4 |
| PDGFR | Platelet-Derived Growth Factor |
| FPR | fMLP |
| FPRL1 | W-tides |
| CRTH2 | prostaglandin D2 |
| C3aR | C3a |
| C5aR | C5a |
| Noci-R | Nociceptin |
| EDG family | Sphingosine 1-phosphate |
| CB1 | Cannabinoids |
| VEGFR | Vascular endothelial growth factor |
| EGFR | Epidermal growth factor |
| FGFR | Fibroblast growth factor |
| P2Y receptor | P2Y |
| CTR | Calcitonin |
| CRLR | Calcitonin gene-related peptide (CGRP) |
| Histamine receptor | Histamine |
| Thrombin receptor | Thrombin |
| TrkB | Brain-derived neurotrophic factor (BDNF) |
| TxA₂ (TP) | Thromboxane A₂ (TxA₂) |
| PGI₂ (IP) | Prostacycline (PGI₂) |

¹This list of chemoattractant receptors is not meant to be exhaustive.
²Only examples of some ligands for each receptor are given. This list is not meant to be exhaustive.

Chemoattractants also include a group of molecules named chemokines. Chemokines, also known as “intercrines” and “SIS cytokines,” comprise a family of more than 50 small secreted proteins (e.g., 70-100 amino acids and about 8-10 kiloDaltons) that play important roles in inflammatory responses, leukocyte trafficking, angiogenesis, and other biological processes related to the migration and activation of cells. As mediators of chemotaxis and inflammation, chemokines play roles in pathological conditions.

The name “chemokine” is derived from chemotactic cytokine, and refers to the ability of these proteins to stimulate chemotaxis (i.e., cell migration) of leukocytes. Indeed, chemokines may comprise the main attractants for inflammatory cells into pathological tissues. See generally, Baggiolini et al., Annu. Rev. Immunol, 15: 675-705 (1997); and Baggiolini et al., Advances in Immunology, 55:97-179 (1994).

Known chemokines are typically assigned to one of four subfamilies based on the arrangement of cysteine motifs. In the so-called alpha-chemokines, for example, the first two of four cysteines (starting from the amino terminus) are separated by an intervening amino acid (i.e., having the motif C-X-C). The beta-chemokines are characterized by the absence of an intervening amino acid between first two cysteines (i.e., comprising the motif C-C). The smaller gamma- and delta-chemokine families are characterized by a single C residue or a pair of cysteines separated by three residues, respectively. For reviews on chemokines, see Ward et al., 1998, Immunity 9:1-11 and Baggiolini et al., 1998, Nature 392:565-568, and the references cited therein.

Examples of chemokines include, but are not limited to, IL-8, GCP-2, Gro α, Gro β, Gro γ, ENA-78, PBP, MIG, IP-10, I-TAC, SDF-1α (PBSF), BLC (BCA-1), MIP-1α, MIP-1β, RANTES, HCC-1, -2, -3, and -4, MCP-1, -2, -3, and -4, eotaxin-1, eotaxin-2, TARC, MDC, MIP-3α (LARC), MIP-3β (ELC), 6Ckine (LC), I-309, TECK, lymphotactin, fractalkine (neurotactin), TCA-4, Exodus-2, Exodus-3 and CKβ-11 (TABLE 3).

Chemokine activity may be mediated by receptors, which are termed “chemokine receptors.” For example, several seven-transmembrane-domain G protein-coupled receptors for C-C chemokines have been cloned: a C-C chemokine receptor-1 (CCR1) which recognizes MIP-1α, RANTES, MCP-2, MCP-3, and MIP-5 (Neote et al., 1993, Cell, 72:415-415); CCR2 which is a receptor for MCP1, 2, 3, 4 and 5; CCR3 which is a receptor for RANTES, MCP-2, 3, 4, MIP-5 and eotaxin; CCR5 which is a receptor for MIP-1α, MIP-1β and RANTES; CCR4 which is a receptor for MDC and TARC; CCR6 which is a receptor for LARC; and CCR7 which is a receptor for SLC and ELC (MIP-3β; reviewed in Sallusto et al., 1998, Immunol. Today 19:568 and Ward et al., 1998, Immunity 9:1-11).

Examples of chemokine receptors include, but are not limited to, the CXC class, e.g., CXCR1, CXCR2, CXCR3, CXCR4, CXCR5; the CC class, CCR1, CCR2, CCR3, CCR4, CCR5, CCR6, CCR7, CCR8, CCR9, CCR10 and CCR11; the CX3CR class, such as CX3CR1 and the XCR class, such as XCR1.

Exemplary chemokine receptors and their ligands are summarized in TABLE 3 below.

TABLE 3
Summary of the known chemokine receptors and some of
their known human ligands (Rossi and Zlotnik, 2000)

| Receptor | Human ligands |
|---|---|
| CXCR1 | IL-8, GCP-2 |
| CXCR2 | IL-8, GCP-2, Gro α, Gro β, Gro γ, ENA-78, PBP |
| CXCR3 | MIG, IP-10, I-TAC |
| CXCR4 | SDF-1α/PBSF |
| CXCR5 | BLC/BCA-1 |
| CCR1 | MIP-1α, MIP-1β, RANTES, HCC-1, 2, 3, and 4 |
| CCR2 | MCP-1, MCP-2, MCP-3, MCP-4 |
| CCR3 | eotaxin-1, eotaxin-2, MCP-3 |
| CCR4 | TARC, MDC, MIP-1α, RANTES |
| CCR5 | MIP-1α, MIP-1β, RANTES |
| CCR6 | MIP-3α/LARC |
| CCR7 | MIP-3β/ELC, 6Ckine/LC |
| CCR8 | I-309 |
| CCR9 | TECK |
| XCR1 | Lymphotactin |
| CX3CR1 | Fractalkine/neurotactin |
| CXCR6 | CXCL16 |
| CXCR7 (RDC-1) | SDF-1, I-TAC |
| CCR10 | CTACK |

On binding their cognate ligands, chemokine receptors transduce an intracellular signal though the associated trimeric G protein, resulting in a rapid increase in intracellular calcium concentration.

Chemokine activity may also be mediated by the unrelated chemotactic peptide receptors N-formyl peptide receptor (fMLP-R or FPR) and its homologues the ‘orphan’ receptors FPRL1 and FPRL2 (U.S. Publication No. 2006-0034863 A1; Bao, L., et al. Genomics 13, 437-40 (1992); Murphy, P. M. et al., J Biol Chem 267, 7637-43 (1992); Durstin, M., et al., Biochem Biophys Res Commun 201, 174-9 (1994); Yang, D., et al, J Immunol 166, 4092-8 (2001)) (reviewed in Le, Y., Murphy, P. M. & Wang, J. M. Formyl-peptide receptors revisited. Trends Immunol 23, 541-8 (2002).

FPRL1 was originally cloned from a human phagocyte cDNA library and was characterized by nucleotide homology to FPR, although FPRL1 interacts very weakly with fMLP, the main ligand for FPR (Bao, L., et al., Genomics 13, 437-40 (1992); Murphy, P. M. et al., J Biol Chem 267, 7637-43 (1992)). FPRL1 receptors induce chemotaxis, but can also activate myeloid cells and thereby stimulate their antigen-presenting properties (Le, Y., Murphy, P. M. & Wang, J. M. Trends Immunol 23, 541-8 (2002); Le, Y. et al., J Neurosci 21, RC123 (2001); and Le, Y., et al., Cytokine Growth Factor Rev 12, 91-105 (2001)). FPRL1 has also been reported to act as a functional lipoxin A4 receptor (Fiore, S. & Serhan, C. N., Biochemistry 34, 16678-86 (1995); Fiore, S., et al. J Exp Med 180, 253-60 (1994); Levy, B. D. et al. Nat Med 8, 1018-23 (2002); and Macphee, C. H. et al., J Immunol 161, 6273-9 (1998)), although there is still some debate over this activity. More recently, several groups studying FPRL1 have described a broad spectrum of low-affinity pathogen-related peptide and lipid ligands as well as several high affinity, but non-natural, synthetic peptide ligands (Le, Y., et al., Trends Immunol 23, 541-8 (2002); Le, Y. et al., J Neurosci 21, RC123 (2001); Le, Y., et al., Cytokine Growth Factor Rev 12, 91-105 (2001); Fiore, S. & Serhan, C. N. Biochemistry 34, 16678-86 (1995); Fiore, S., et al., J Exp Med 180, 253-60 (1994); Le, Y. et al., J Immunol 166, 1448-51 (2001); and Le, Y. et al., J Immunol 163, 6777-84 (1999)). The ability of FPRL1 to interact with this broad spectrum of pathogen-related ligands (TABLE 4, from Le, Y., et al., Cytokine and Growth Factor Reviews 12:91-105 (2001)) is unusual amongst G protein-coupled receptors (GPCRs) and suggests that FPRL1 may represent a novel type of pattern recognition receptors (PRR) with the potential for regulating innate immune responses to a number of viral and bacterial pathogens.

TABLE 4
Agonists and antagonists of formyl peptide receptors.

| LIGANDS | FPR | FPRL1 |
|---|---|---|
| Agonists | | |
| Bacterial peptide | | |
| fMLF | ++++ | + |
| H. pylori peptide, Hp(2-20) | − | +++ |
| HIV-1 Env domains: | | |
| T20/DP176 | ++++ | + |
| T21/DP107 | +++ | ++++ |
| N36 | − | +++ |
| F peptide | − | +++ |
| V3 peptide | − | +++ |
| Host-derived agonists: | | |
| LL-37 | − | ++++ |
| SAA | − | ++++ |
| Aβ₄₂ | ++ | ++++ |
| PrP106-126 | − | ++++ |
| Annexin I | +++ | +++ |
| Mitochondrial peptide | − | ++++ |
| LXA4 | − | ++++ |
| Humanin | − | ++ |
| Temporin A | − | ++ |
| Peptide library derived agonists | +++ | ++++ |
| W peptide | − | ++++ |
| MMK-1 | + | +++ |
| Quinazolinone-C1 | | |
| Antagonists: | | |
| Boc-FLFLF | ++ | ? |
| CsH | +++ | − |
| Deoxycholoc acid (DCA) | +++ | +++ |
| Chenodeoxycholic acid (CDCA) | +++ | +++ |

In addition to the agonists and antagonists shown in TABLE 4, SHAAGtide polypeptides were also shown to modulate FPRL1 activity. The term “SHAAGtide” includes SHAAGtide polypeptides (shown in TABLE 5), including isolated or purified SHAAGtide polypeptides, proteins or biologically active fragments separated from or recovered from a component of its natural environment; and polynucleotides shown in TABLE 6, truncated variants and other variants thereof, including naturally occurring allelic variants, derivatives and analogs. SHAAGtide polypeptides are derived from the alternatively spliced variant of CKβ8, namely CKβ8-1. CKβ8-1 is a 116 amino acid long polypeptide that includes the sequence of SHAAGtide polypeptides.

“SHAAGtide variant” means an active SHAAGtide polypeptide having at least: (1) about 80% amino acid sequence identity with a full-length native sequence SHAAGtide polypeptide sequence or (2) any fragment of a full-length SHAAGtide polypeptide sequence. For example, SHAAGtide polypeptide variants include SHAAGtide polypeptides wherein one or more amino acid residues are added or deleted at the N- or C-terminus of the full-length native amino acid sequence, with the exception of those fragments that are identical to CKβ8 and CKβ8-1. A SHAAGtide polypeptide variant will have at least about 80% amino acid sequence identity, preferably at least about 81% amino acid sequence identity, more preferably at least about 82%, 83%, 84%, 85%, 86%, 87%, 88%, 89%, 90%, 91%, 92%, 93%, 94%, 95%, 96%, 97%, 98% amino acid sequence identity and most preferably at least about 99% amino acid sequence identity with a full-length native sequence SHAAGtide polypeptide sequence. Ordinarily, SHAAGtide variant polypeptides are at least about 10 amino acids in length, often at least about 20 amino acids in length, more often at least about 30, 40, 50, 60, 70, 80, 90, 100, 150, 200, or 300 amino acids in length, or more. The SHAAGtide variant polypeptides may be in a range from about 10 amino acids to about 300 amino acids or more. Preferably, the SHAAGtide variant polypeptides are in a range from about 10 amino acids to about 100 amino acids; more preferably in a range from about 10 amino acids to about 90 amino acids. Any ranges between about 10 to about 90 amino acids are also contemplated. For example, the SHAAGtide variant polypeptide may be in a range from about 10 amino acids to about 20, 30, 40, 50, 60, 70, and 80 amino acids.

In general, a SHAAGtide polypeptide variant that preserves SHAAGtide polypeptide-like function and includes any variant in which residues at a particular position in the sequence have been substituted by other amino acids, and further includes the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

SHAAGtides were previously shown to act as adjuvants to enhance the immune response to an immunogen or antigen, for example, in U.S. patent application Ser. No. US 2006-0034863 A1, disclosure of which is incorporated by reference herein in its entirety.

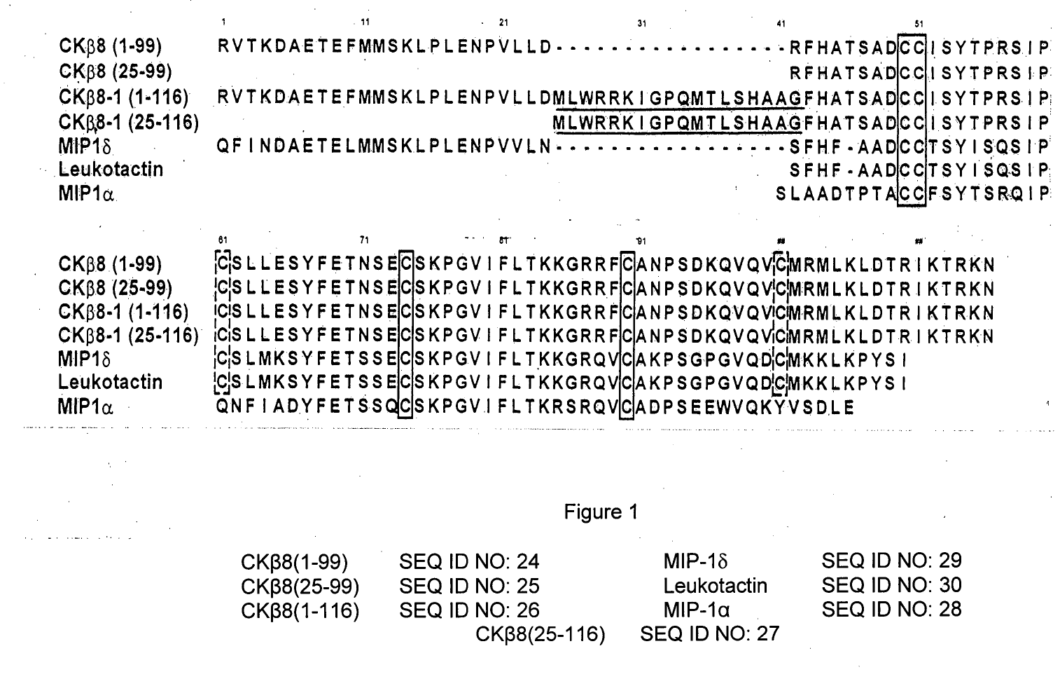

TABLE 5 shows the SHAAGtide polypeptide sequence (SEQ ID NO: 1) and the polypeptide sequences of certain SHAAGtide truncated variants and other variants. TABLE 6 shows the SHAAGtide polynucleotide sequence (SEQ ID NO: 12) and the polynucleotide sequences of SHAAGtide truncated variants and other variants. TABLE 7 shows the human CKβ8-1(25-116) nucleotide sequence (SEQ ID NO: 23). FIG. 1 shows the amino acid sequence alignment of the human CKβ8 variants (CKβ (1-99)—SEQ ID NO: 24; CKβ (25-99)—SEQ ID NO: 25; CKβ (1-116)—SEQ ID NO: 26; CKβ (25-116)—SEQ ID NO: 27) with human CCL15/MIP-1α (SEQ ID NO: 28); CCL3/MIP-1δ (SEQ ID NO: 29) and Leukotactin (SEQ ID NO: 30). Four conserved cysteine residues are shown in boxes and two additional cysteines, not normally found in the CC chemokine family, are shown in dashed boxes. The alternatively spliced exon of CCL23/CKβ8-1 is shown underlined.

TABLE 5

SHAAGtide and various truncated and other
variants - amino acid sequences.

| SEQ | Designation | |
|---|---|---|
| ID | and FPRL1 | |
| NO: | Activity | Amino acid sequence |
| 1 | CCXP1 | Met Leu Trp Arg Arg Lys Ile Gly |
| | Native | 1 5 |
| | sequence high | Pro Gln Met Thr Leu Ser His Ala |
| | activity | 10 15 |
| | | Ala Gly |
| 2 | CCXP2 | Arg Arg Lys Ile Gly Pro Gln Met |
| | Low | 1 5 |
| | activity | Thr Leu Ser His Ala Ala Gly |
| | | 10 15 |
| 3 | CCXP3 | Met Leu Trp Arg Arg Lys Ile Gly |
| | High | 1 5 |
| | activity | Pro Gln Met Thr Leu Ser His |
| | | 10 15 |
| 4 | CCXP4 | Ile Gly Pro Gln Met Thr Leu Ser |
| | Low | 1 5 |
| | activity | His Ala Ala Gly |
| | | 10 |
| 5 | CCXP5 | Met Leu Trp Arg Arg Lys Ile Gly |
| | Moderate | 1 5 |
| | activity | Pro Gln Met Thr |
| | | 10 |
| 6 | CCXP6 | Met Leu Trp Arg Arg Lys Ile Gly |
| | high | 1 5 |
| | activity | Pro Gln Met Thr Leu Ser His Ala |
| | | 10 15 |
| | | Ala Tyr |
| 7 | CCXP7 | Trp Arg Arg Lys Ile Gly Pro Gln |
| | Low activity | 1 5 |
| | | Met Thr Leu Ser His Ala Ala Gly |
| | | 10 15 |
| 8 | CCXP8 | Met Leu Trp Arg Arg Lys Ile Gly |
| | Moderate | 1 5 |
| | activity | Pro Gln Met |
| | | 10 |
| 9 | CCXP9 | Trp Arg Arg Lys Ile Gly Pro Gln |
| | Low activity | 1 5 |
| | | Met |
| 10 | CCXP10 | Trp Arg Arg Lys Ile Gly |
| | Low activity | 1 5 |
| 11 | CCXP11 | Leu Trp Arg Arg Lys Ile Gly Pro |
| | Moderate | 1 5 |
| | activity | Gln Met Thr Leu Ser His |
| | | 10 |

TABLE 6

SHAAGtide and various truncated and other
variants - polynucleotide sequences

| | |
|---|---|
| SEQ | |
| ID | |
| NO: | Polynucleotide sequence |

| | | |
|---|---|---|
| 12 | atgctctgga ggagaaagat tggtcctcag atgacccttt | 54 |
| | ctcatgctgc agga | |
| 13 | aggagaaaga ttggtcctca gatgaccctt tctcatgctg | 45 |
| | cagga | |
| 14 | atgctctgga ggagaaagat tggtcctcag atgacccttt | 45 |
| | ctcat | |
| 15 | attggtcctc agatgaccct ttctcatgct gcagga | 36 |
| 16 | atgctctgga ggagaaagat tggtcctcag atgacc | 36 |
| 17 | atgctctgga ggagaaagat tggtcctcag atgacccttt | 54 |
| | ctcatgctgc atat | |
| 18 | tggaggagaa agattggtcc tcagatgacc ctttctcatg | 48 |
| | ctgcagga | |
| 19 | atgctctgga ggagaaagat tggtcctcag atg | 33 |
| 20 | tggaggagaa agattggtcc tcagatg | 27 |
| 21 | tggaggagaa agattggt | 18 |
| 22 | ctctggagga gaaagattgg tcctcagatg accctttctc | 42 |
| | at | |

TABLE 7

Human CKβ8-1(25-116) nucleotide sequence
(SEQ ID NO: 23)

| | |
|---|---|
| atgctctgga ggagaaagat tggtcctcag atgacccttt | 60 |
| ctcatgctgc aggattccat | |
| gctactagtg ctgactgctg catctcctac accccacgaa | 120 |
| gcatcccgtg ttcactcctg | |
| gagagttact ttgaaacgaa cagcgagtgc tccaagccgg | 180 |
| gtgtcatctt cctcaccaag | |
| aaggggcgac gtttctgtgc caaccccagt gataagcaag | 240 |
| ttcaggtttg catgagaatg | |
| ctgaagctgg acacacggat caagaccagg aagaattga | 279 |

In addition to SHAAGtide polypeptide, W-tides can also act as ligands to a chemoattractant receptor, such as FPRL1, causing intracellular calcium flux in leukocytes and inducing chemotactic migration of human monocytes. In addition to monocytes, W-tides effectively attract other types of leukocytes, namely neutrophils.

W-tides were previously shown to function as adjuvants, for example in U.S. Publication No. 2005-0234004 A1, disclosure of which is incorporated herein in its entirety.

At least twenty eight W-tides have been identified and described. Examples of W-tides and protein and peptides comprising W-tide sequences that may be used with the present invention include, but are not limited to, the following W-tides. W-tides such as HFYLPM (SEQ ID NO: 31) and MFYLPM (SEQ ID NO: 32) were identified by Bae et al., 2001). Additionally, examples of W-tides include the synthetic hexapeptides HFYLPm (SEQ ID NO: 33) and WKYMVm (SEQ ID NO: 34) (Baek SH et al., 1996). A publication, WO 03/064447 A2, further identifies twenty four variants of WKYMVm polypeptide, SEQ ID NOS: 31-58, which may be used with the present invention and which are included in TABLE 8, below. Standard amino acid abbreviations are used in the TABLE 8; lower case letters identify D-residues.

TABLE 8
Exemplary W-tide polypeptide sequences.

| | |
|---|---|
| SEQ ID NO: | Amino acid sequence |

| | |
|---|---|
| 31 | His-Phe-Tyr-Leu-Pro-Met-CONH₂; HFYLPM |
| 32 | Met-Phe-Tyr-Leu-Pro-Met-CONH₂; MFYLPM |
| 33 | His-Phe-Tyr-Leu-pro-D-Met-CONH₂; HFYLPm |
| 34 | Trp-Lys-Tyr-Met-Val-D-Met-CONH₂; WKYMVm |
| 35 | Trp-Lys-Gly-Met-Val-D-Met-NH₂; WKGMVm |
| 36 | Trp-Lys-Tyr-Met-Gly-D-Met-NH₂; WKYMGm |
| 37 | Trp-Lys-Tyr-Met-Val-Gly-NH₂; WKYMVG |
| 38 | Trp-kg-Tyr-Met-Val-D-Met-NH₂; WRYMVm |
| 39 | Trp-Glu-Tyr-Met-Val-D-Met-NH₂; WEYMVm |
| 40 | Trp-His-Tyr-Met-Val-D-Met-NH₂; WHYMVm |
| 41 | Trp-Asp-Tyr-Met-Val-D-Met-NH₂; WDYMVm |
| 42 | Trp-Lys-His-Met-Val-D-Met-NH₂; WKHMVm |
| 43 | Trp-Lys-Glu-Met-Val-D-Met-NH₂; WKEMVm |
| 44 | Trp-Lys-Trp-Met-Val-D-Met-NH₂; WKWMVm |
| 45 | Trp-Lys-Arg-Met-Val-D-Met-NH₂; WKRMVm |
| 46 | Trp-Lys-Asp-Met-Val-D-Met-NH₂; WKDMVm |
| 47 | Trp-Lys-Phe-Met-Val-D-Met-NH₂; WKFMVm |
| 48 | Trp-Lys-Tyr-Met-Tyr-D-Met-NH₂; WKYMYm |
| 49 | Trp-Lys-Tyr-Met-(Phe/Trp)-D-Met-NH₂; |
| | WKYM(F/W)m |
| 50 | Trp-Lys-Tyr-Met-Val-Glu-NH₂; WKYMVE |
| 51 | Trp-Lys-Tyr-Met-Val-Val-NH₂; WKYMVV |
| 52 | Trp-Lys-Tyr-Met-Val-Arg-NH₂; WKYMVR |
| 53 | Trp-Lys-Tyr-Met-Val-Trp-NH₂; WKYMVW |
| 54 | Trp-Lys-Tyr-Met-Val-NH₂; WKYMV |
| 55 | Lys-Tyr-Met-Val-D-Met-NH₂; KYMVm |
| 56 | Lys-Tyr-Met-Val-NH₂; KYMV |
| 57 | Tyr-Met-Val-D-Met-NH₂; YMVm |
| 58 | Met-Val-D-Met-NH₂; MVm |

Any of the described chemoattractants may be used in compositions of this invention to enhance adjuvant activity of ligand for TLR to enhance an immune response to an antigen and immunogen.

IV. Exemplary Antigens and Immunogens

As used herein, the term “antigen” refers to a molecule which contains one or more epitopes capable of stimulating a host's immune system to make a cellular antigen-specific immune response when the antigen is presented in accordance with the present invention, or a humoral antibody response. An “immunogen” is any substance or organism that provokes immune response (produces immunity when introduced into a subject). An antigen or immunogen may be capable of eliciting a cellular or humoral response by itself or when present in combination with another molecule. Normally, an epitope will include between about 3-15, preferably about 5-15, and more preferably about 7-15 amino acids. Epitopes of a given protein can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715, all incorporated herein by reference in their entireties. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. The term “antigen” as used herein denotes both subunit antigens, i.e., antigens which are separate and discrete from a whole organism with which the antigen is associated in nature, as well as killed, attenuated, disrupted or inactivated bacteria, viruses, parasites or other microbes. Similarly, an oligonucleotide or polynucleotide which expresses a therapeutic or immunogenic protein, or antigenic determinant in vivo, such as in gene therapy and nucleic acid immunization applications, is also included in the definition of antigen herein. Further, for purposes of the present invention, antigens may be derived from any of several known viruses, bacteria, parasites and fungi, as well as any of the various tumor antigens. Furthermore, for purposes of the present invention, an “antigen” refers to a protein which includes modifications, such as deletions, additions and substitutions (generally conservative in nature), to the native sequence, as long as the protein maintains the ability to elicit an immunological response. These modifications may be deliberate, as through site-directed mutagenesis, or may be accidental, such as through mutations of hosts which produce the antigens.

Antigenic or immunogenic agents that may be used in the immunogenic compositions of the invention include antigens from an animal, a plant, a bacteria, a protozoan, a parasite, a virus or a combination thereof. The antigenic or immunogenic agent may be any viral peptide, protein, polypeptide, or a fragment thereof derived from a virus including, but not limited to, RSV-viral proteins, e.g., RSV F glycoprotein, RSV G glycoprotein, influenza viral proteins, e.g., influenza virus neuraminidase, influenza virus hemagglutinin, avian flu H5N1 strain HA antigens, herpes simplex viral protein, e.g., herpes simplex virus glycoprotein including for example, gB, gC, gD, and gE. The antigenic or immunogenic agent for use in the compositions of the invention may be an antigen of a pathogenic virus such as, an antigen of adenovirdiae (e.g., mastadenovirus and aviadenovirus), herpesviridae (e.g., herpes simplex virus 1, herpes simplex virus 2, herpes simplex virus 5, and herpes simplex virus 6), leviviridae (e.g., levivirus, enterobacteria phase MS2, allolevirus), poxyiridae (e.g., chordopoxvirinae, parapoxvirus, avipoxvirus, capripoxvirus, leporipoxvirus, suipoxvirus, molluscipoxvirus, and entomopoxvirinae), papovaviridae (e.g., polyomavirus and papillomavirus), paramyxoviridae (e.g., paramyxovirus, parainfluenza virus 1, mobillivirus (e.g., measles virus), rubulavirus (e.g., mumps virus), pneumonovirinae (e.g., pneumovirus, human respiratory syncytial virus), metapneumovirus (e.g., avian pneumovirus and human metapneumovirus), picornaviridae (e.g., enterovirus, rhinovirus, hepatovirus (e.g., human hepatitis A virus), cardiovirus, and apthovirus, reoviridae (e.g., orthoreovirus, orbivirus, rotavirus, cypovirus, fijivirus, phytoreovirus, and oryzavirus), retroviridae (e.g., mammalian type B retroviruses, mammalian type C retroviruses, avian type C retroviruses, type D retrovirus group, BLV-HTLV retroviruses), lentivirus (e.g. human immunodeficiency virus 1 and human immunodeficiency virus 2), spumavirus, flaviviridae (e.g., hepatitis C virus), hepadnaviridae (e.g., hepatitis B virus), togaviridae (e.g., alphavirus (e.g., sindbis virus) and rubivirus (e.g., rubella virus), rhabdoviridae (e.g., vesiculovirus, lyssavirus, ephemerovirus, cytorhabdovirus, and necleorhabdovirus), arenaviridae (e.g., arenavirus, lymphocytic choriomeningitis virus, lppy virus, and lassa virus), and coronaviridae (e.g., coronavirus and torovirus).

Alternatively, the antigenic or immongenic agent in the immunogenic compositions of the invention may be a cancer or tumor antigen including but not limited to, KS 1/4 pan-carcinoma antigen, ovarian carcinoma antigen (CA125), prostatic acid phosphate, prostate specific antigen, melanoma-associated antigen p97, melanoma antigen gp75, high molecular weight melanoma antigen (HMW-MM), prostate specific membrane antigen, carcinoembryonic antigen (CEA), polymorphic epithelial mucin antigen, human milk fat globule antigen, colorectal tumor-associated antigens such as: CEA, TAG-72, CO17-1A; GICA 19-9, CTA-1 and LEA, Burkitt's lymphoma antigen-38.13, CD19, human B-lymphoma antigen-CD20, CD33, melanoma specific antigens such as ganglioside GD2, ganglioside GD3, ganglioside GM2, ganglioside GM3, tumor-specific transplantation type of cell-surface antigen (TSTA) such as virally-induced tumor antigens including T-antigen DNA tumor viruses and Envelope antigens of RNA tumor viruses, oncofetal antigen-alpha-fetoprotein such as CEA of colon, bladder tumor oncofetal antigen, differentiation antigen such as human lung carcinoma antigen L6, L20, antigens of fibrosarcoma, human leukemia T cell antigen-Gp37, neoglycoprotein, sphingolipids, breast cancer antigen such as EGFR (Epidermal growth factor receptor), HER2 antigen (p185^{HER2}), polymorphic epithelial mucin (PEM), malignant human lymphocyte antigen-APO-1, differentiation antigen such as I antigen found in fetal erythrocytes, primary endoderm, I antigen found in adult erythrocytes, preimplantation embryos, I(Ma) found in gastric adenocarcinomas, M18, M39 found in breast epithelium, SSEA-1 found in myeloid cells, VEP8, VEP9, Myl, VIM-D5, D₁56-22 found in colorectal cancer, TRA-1-85 (blood group H), C14 found in colonic adenocarcinoma, F3 found in lung adenocarcinoma, AH6 found in gastric cancer, Y hapten, Le^{y} found in embryonal carcinoma cells, TL5 (blood group A), EGF receptor found in A431 cells, E₁ series (blood group B) found in pancreatic cancer, FC10.2 found in embryonal carcinoma cells, gastric adenocarcinoma antigen, CO-514 (blood group Le^{a}) found in Adenocarcinoma, NS-10 found in adenocarcinomas, CO-43 (blood group Le^{b}), G49 found in EGF receptor of A431 cells, MH2 (blood group ALe^{b}/Le^{y}) found in colonic adenocarcinoma, 19.9 found in colon cancer, gastric cancer mucins, T₅A₇found in myeloid cells, R₂₄ found in melanoma, 4.2, G_{D3}, D1.1, OFA-1, G_{M2}, OFA-2, GD2, and M1:22:25:8 found in embryonal carcinoma cells, and SSEA-3 and SSEA-4 found in 4 to 8-cell stage embryos, and a T cell receptor derived peptide from a Cutaneous T cell Lymphoma.

The antigenic or immunogenic agent used in the immunogenic composition of this invention may be an infectious disease agent including, but not limited to, influenza virus hemagglutinin (Genbank Accession No. JO2132; Air, 1981, Proc. Natl. Acad. Sci. USA 78: 7639-7643; Newton et al., 1983, Virology 128: 495-501), avian flu H5N1 strain HA antigens, human respiratory syncytial virus G glycoprotein (Genbank Accession No. Z33429; Garcia et al., 1994, J. Virol.; Collins et al., 1984, Proc. Natl. Acad. Sci. USA 81: 7683), core protein, matrix protein or any other protein of Dengue virus (Genbank Accession No. M19197; Hahn et al., 1988, Virology 162: 167-180), measles virus hemagglutinin (Genbank Accession No. M81899; Rota et al., 1992, Virology 188: 135-142), herpes simplex virus type 2 glycoprotein gB (Genbank Accession No. M14923; Bzik et al., 1986, Virology 155:322-333), poliovirus I VP1 (Emini et al., 1983, Nature 304:699), envelope glycoproteins of HIV I (Putney et al., 1986, Science 234: 1392-1395), hepatitis B surface antigen (Itoh et a., 1986, Nature 308: 19; Neurath et al., 1986, Vaccine 4: 34), diptheria toxin (Audibert et al., 1981, Nature 289: 543), streptococcus 24M epitope (Beachey, 1985, Adv. Exp. Med. Biol. 185:193), gonococcal pilin (Rothbard and Schoolnik, 1985, Adv. Exp. Med. Biol. 185:247), pseudorabies virus g50 (gpD), pseudorabies virus II (gpB), pseudorabies virus gIII (gpC), pseudorabies virus glycoprotein H, pseudorabies virus glycoprotein E, transmissible gastroenteritis glycoprotein 195, transmissible gastroenteritis matrix protein, swine rotavirus glycoprotein 38, swine parvovirus capsid protein, Serpulina hydodysenteriae protective antigen, bovine viral diarrhea glycoprotein 55, Newcastle disease virus hemagglutinin-neuraminidase, swine flu hemagglutinin, swine flu neuraminidase, foot and mouth disease virus, hog cholera virus, swine influenza virus, African swine fever virus, Mycoplasma hyopneumoniae, infectious bovine rhinotracheitis virus (e.g., infectious bovine rhinotracheitis virus glycoprotein E or glycoprotein G), or infectious laryngotracheitis virus (e.g., infectious laryngotracheitis virus glycoprotein G or glycoprotein 1), a glycoprotein of La Crosse virus (Gonzales-Scarano et al., 1982, Virology 120: 42), neonatal calf diarrhea virus (Matsuno and Inouye, 1983, Infection and Immunity 39: 155), Venezuelan equine encephalomyelitis virus (Mathews and Roehrig, 1982, J. Immunol. 129: 2763), punta toro virus (Dalrymple et al., 1981, in Replication of Negative Strand Viruses, Bishop and Compans (eds.), Elsevier, N.Y., p. 167), murine leukemia virus (Steeves et al., 1974, J. Virol. 14:187), mouse mammary tumor virus (Massey and Schochetman, 1981, Virology 115: 20), hepatitis B virus core protein and/or hepatitis B virus surface antigen or a fragment or derivative thereof (see, e.g., U.K. Patent Publication No. GB 2034323A published Jun. 4, 1980; Ganem and Varmus, 1987, Ann. Rev. Biochem. 56:651-693; Tiollais et al., 1985, Nature 317:489-495), antigen of equine influenza virus or equine herpesvirus (e.g., equine influenza virus type A/Alaska 91 neuraminidase, equine influenza virus type A/Miami 63 neuraminidase, equine influenza virus type A/Kentucky 81 neuraminidase equine herpesvirus type 1 glycoprotein B, and equine herpesvirus type 1 glycoprotein D, antigen of bovine respiratory syncytial virus or bovine parainfluenza virus (e.g., bovine respiratory syncytial virus attachment protein (BRSV G), bovine respiratory syncytial virus fusion protein (BRSV F), bovine respiratory syncytial virus nucleocapsid protein (BRSV N), bovine parainfluenza virus type 3 fusion protein, and the bovine parainfluenza virus type 3 hemagglutinin neuraminidase), bovine viral diarrhea virus glycoprotein 48 or glycoprotein 53.

The antigenic or immunogenic agent for use in the immunogenic compositions of the invention may be any substance that under appropriate conditions results in an immune response in a subject, including, but not limited to, polypeptides, peptides, proteins, glycoproteins, lipids, nucleic acids and polysaccharides. The concentration of the antigenic or immunogenic agent in the immunogenic compositions of the invention may be determined using standard methods known to one skilled in the art and depends on chemical nature and the potency of the antigenic or immunogenic agent. Typically, the starting concentration of the antigenic or immunogenic agent in the composition of this invention is the amount that is conventionally used for eliciting the desired immune response. The concentration of the antigenic or immunogenic agent in the composition of this invention is then adjusted, e.g., by dilution using a diluent, so that an effective protective immune response is achieved as assessed using standard methods known in the art and described herein. The concentration of the antigenic or immunogenic agent is preferably less than the conventional amounts used.

The immunogenic composition of this invention may comprise one or more antigenic or immunogenic agents.

VI. Immunogenic Compositions

In one embodiment, present invention relates to compositions for inducing immunity, including mucosal immunity. The composition includes an antigenic or immunogenic agent, a ligand for a TLR and at least one chemoattractant. The composition includes effective amounts for inducing an adjuvant response (i.e., immunity, mucosal immunity) of a combination of adjuvants, wherein the combination of adjuvants includes a ligand for TLR, such as a CpG, and at least one chemoattractant.

The immunogenic compositions of the invention comprise an antigenic or immunogenic agent, and a combination of adjuvants, including a ligand for a TLR, and at least one chemoattractant, wherein the combination of the adjuvants enhances the presentation and/or availability of the antigenic or immunogenic to an immune cell, resulting in an enhanced immune response. The immunogenic compositions of the invention may enhance cell-mediated and/or humoral mediated immune response. Cell-mediated immune responses that may be modulated by the intradermal vaccine formulations of the invention include for example, Th1 or Th2 CD4+ T-helper cell-mediated or CD8+ cytotoxic T-lymphocytes mediates responses.

The immunogenic composition of this invention may further include pharmaceutically acceptable excipients and carriers. An excipient is a more or less inert substance added in a composition as a diluent or vehicle. Alternatively, an excipient may be used to give form or consistency to a composition. Excipients that may be used in the immunogenic compositions of this invention include, but are not limited to, stabilizers, preservatives, solvents, surfactants or detergents, suspending agents, tonicity agents, a muco or bioadhesive, and vehicles and ingredients for growth medium. Examples of excipients are known to one skilled in the art and encompassed within the instant invention, see, e.g., Remington's Pharmaceutical Sciences Mack Pub. Co., N.J., current edition; all of which is incorporated herein by reference in its entirety. Excipients may be used in the preparation and manufacturing of immunogenic compositions. In such cases, residual concentrations of the excipient may be found in the final immunogenic composition, left over from the manufacturing or preparation of the composition. Such residual concentrations are too low to result in the adjuvant activity observed with the immunogenic compositions of the invention.

Preferably, the pharmaceutically acceptable carrier does not itself induce a physiological response, e.g., an immune response. Most preferably, the pharmaceutically acceptable carrier does not result in any adverse or undesired side effects and/or does not result in undue toxicity. Pharmaceutically acceptable carriers for use in the immunogenic compositions of the invention include, but are not limited to, saline, buffered saline, dextrose, water, glycerol, sterile isotonic aqueous buffer, and combinations thereof.

In particular embodiments, the immunogenic compositions of the invention, may also contain wetting agents, emulsifying agents, or pH buffering agents. The immunogenic compositions of the invention may be a solid, such as a lyophilized powder suitable for reconstitution, a liquid solution, a suspension, a tablet, a pill, a capsule, a sustained release formulation, or a powder.

The immunogenic compositions of the invention may be in any form suitable for delivery. Preferably, the immunogenic compositions of the invention are stable formulations, i.e., undergo minimal to no detectable level of degradation and/or aggregation of the antigenic or immunogenic agent, and may be stored for an extended period of time (hours, days, months, or years) with no loss in biological activity, e.g., antigenicity or immunogenicity of the antigenic agent.

V. Vaccine Composition

Compositions of this invention may be administered in a form of a vaccine composition. These vaccine compositions may be administered alone or in combinations, e.g., as a complex with cationic liposomes, encapsulated in anionic liposomes, enclosed in chochleates, or they can be encapsulated in microcapsules

Any vaccine composition of this invention may comprise a pharmaceutically acceptable carrier. The pharmaceutically acceptable carrier in the vaccine of the instant invention may comprise saline or other suitable carriers (Arnon, R. (Ed.) Synthetic Vaccines 1:83-92, CRC Press, Inc., Boca Raton, Fla., 1987). The DNA vaccines may be incorporated in liposomes or chocleates to enhance in vivo transfection. Genetic adjuvants, such as immunostimulatory sequences (ISS) and cytokine-encoding nucleic acids may also be employed. See Homer A A et al., 1998, Immunostimulatory DNA Is a Potent Mucosal Adjuvant, Cellular Immunology 190:77-82, and Roman M et al., supra.

VI. Preparation of the Immunogenic Composition

The immunogenic composition of this invention may be prepared by any method that results in a stable, sterile, injectable formulation. Preferably, the method for preparing an immunogenic composition of this invention comprises: providing a solution of the adjuvants; providing a solution of the antigenic or immunogenic agent; and combining the solution of the adjuvants and the solution of the antigenic or immunogenic agent to form the inoculum, e.g., the solution to be injected to the subject.

In one embodiment, the chemoattractant and a ligand for TLR (i.e., adjuvant(s) of this invention), in a particulate form, may be dissolved in a solution of the antigenic or immunogenic agent, such that a stable, sterile, injectable formulation is formed. Alternatively, the antigenic or immunogenic agent may be particulate and dissolved in the adjuvant solution such that a stable, sterile, injectable formulation is formed. For enhanced performance of the immunogenic composition of this invention, the antigenic or immunogenic agent should be uniformly dispersed throughout the composition.

In one embodiment, the adjuvants and the antigenic or immunogenic agent are mixed prior to administration to a subject. Alternatively, the adjuvants and the antigenic or immunogenic agent may be mixed during administration in a delivery device.

The amount of the antigenic or immunogenic agent used in the immunogenic composition of this invention may vary depending on the chemical nature and the potency of the antigenic or immunogenic agent and the specific adjuvants used. Typically, the starting concentration of the antigenic or immunogenic agent in the composition of this invention is the amount that is conventionally used for eliciting the desired immune response, using the conventional routes of administration, e.g., intramuscular injection. The concentration of the antigenic or immunogenic agent is then adjusted, e.g., by dilution using a diluent so that an effective protective immune response is achieved as assessed using standard methods known in the art and described herein.

The amount of the adjuvants used in the immunogenic composition of this invention may vary depending on the chemical nature of the adjuvants and the specific antigenic or immunogenic agent used. One of ordinary skill in the art would appreciate that depending on the individual adjuvants and the antigenic or immunogenic agent, the amount of adjuvants may be adjusted using the methods that are substantially identical to those disclosed above for the determination of an effective amount of the antigenic or immunogenic agent, as well as other methods conventionally known in the art.

The immunogenic compositions of the present invention can be prepared as unit dosage forms. A unit dosage per vial may contain 0.1 mL to 1 mL, preferably 0.1 to 0.5 mL of the formulation. In some embodiments, a unit dosage form of the immunogenic compositions of the invention may contain 50 μL to 100 μL, 150 μL to 200 μL, or 250 μL to 500 μL of the formulation. If necessary, these preparations may be adjusted to a desired concentration by adding a sterile diluent to each vial. The immunogenic compositions of the invention are more effective in eliciting the desired immune response, and thus the total volume for delivery may be less than the volume that is conventionally used.

In some embodiments, the components of the immunogenic compositions of the invention, e.g., the antigenic or immunogenic agent and the adjuvants, are supplied either separately or mixed together in unit dosage form, for example, as a dry lyophilized powder or water free concentrate in a hermetically sealed container such as an ampoule or a sachette indicating the quantity of the active agent, e.g., the antigenic or immunogenic agent. In other embodiments, an ampoule of sterile diluent may be provided so that the components may be mixed prior to administration. In a specific embodiment, the adjuvants may be mixed with the antigenic or immunogenic agent just prior to administration. In another specific embodiment, the adjuvants may be mixed with the antigenic or immunogenic agent in a delivery device during administration.

The invention also provides immunogenic compositions that are packaged in a hermetically sealed container such as an ampoule or a sachette indicating the quantity of the components. In one embodiment, the immunogenic composition is supplied as a liquid, in another embodiment, as a dry sterilized lyophilized powder or water free concentrate in a hermetically sealed container and can be reconstituted, e.g., with water or saline to the appropriate concentration for administration to a subject. In an alternative embodiment, the immunogenic composition is supplied in liquid form in a hermetically sealed container indicating the quantity and concentration of the components. The immunogenic composition of the invention may be prepared by any method that results in a stable, sterile, injectable formulation.

Preferably, the immunogenic compositions of the invention are administered using any standard delivery devices and methods. Exemplary delivery devices were described in U.S. Pat. No. 6,494,865; US Publication Nos. 2002-0095134 A1 and 2002-0156453 A1; all of which are incorporated herein by reference in their entirety. Other delivery devices will be known to those skilled in the art.

In some embodiments, the immunogenic compositions of the invention are administered within 12 hours, preferably within 6 hours, within 5 hours, within 3 hours, or within 1 hour after preparation, for example, after being reconstituted from the lyophilized powder. In a preferred embodiment, the immunogenic compositions of the invention are prepared for administration into a subject immediately prior to the administration, i.e., the antigenic or immunogenic agent is mixed with the adjuvants.

Preferably, the immunogenic compositions of the invention have little or no short term and/or long term toxicity.

VII. Routes and Methods of Administration

Compositions of this invention may be administered to a subject by any of many standard means for administering the particular composition. For example, compositions can be administered orally, sublingually, intraocularly, intranasally, intravenously, by intramuscular injection, subcutaneously, intradermally, by intraperitoneal injection, topically, transdermally, by inhalation, perfusion, by application to mucosal membranes and the like. Parental administration, if used, is generally characterized by injection. Injectables can be prepared in conventional forms, either as liquid solutions or suspensions, solid forms suitable for solution or suspension in liquid prior to injection, or as emulsions, as described herein. Injectables may be administered using a conventional needle and syringe. However, embodiments of the present invention are not necessarily restricted to such routes or method.

Other methods for delivery can include formulation with cationic lipids and liposomes; this can be applicable to either the DNA form or protein form of an antigen or a chemical such as one capable of immune stimulation, for example by induction of an endogenous cytokine. See Pachuk C J et al., 2000, Curr Opin Mol Ther April 2(2): 188-98; Van Slooten M L et al. 2001, Biochim Biophys Acta 1530:134-45; Van Slooten M L et al., 2000, Pharm Res 17:42-48; Lachman L B et a., 1996, Eur Cytokine Netw 7:693-8. Further methods for delivery can include electroporation, cationic microparticles, ultrasonic distribution, and biolistic particle delivery techniques.

The two adjuvants in the composition, namely ligand for TLR and the chemoattractant, may be administered with any or all of the administrations of antigen. For instance the combination of adjuvants may be administered with a priming dose of antigen. In another embodiment the combination of adjuvants is administered with a boost dose of antigen. In some embodiments the subject is administered a priming dose of antigen and the combination of the adjuvants before the boost dose. In yet other embodiments the subject is administered a boost dose of antigen and the combination of the adjuvants after the priming dose.

In one embodiment the two adjuvants are administered simultaneously. In another embodiment the adjuvants are administered sequentially.

The dosage of the immunogenic composition of this invention depends on the antigenic or immunogenic agent in the composition. The dosage of the immunogenic composition may be determined using standard immunological methods known in the art, for example, by first identifying doses effective to elicit a prophylactic or therapeutic immune response, e.g., by measuring the serum titer of antigen specific immunoglobulins, relative to a control formulation, e.g., a formulation simply consisting of the antigenic or immunogenic agent alone or with a ligand for TLR without a chemoattractant adjuvant as disclosed herein. Preferably, the effective dose is determined in an animal model, prior to use in humans.

The immunogenic compositions of this invention may also be administered on a dosage schedule, for example, an initial administration of the immunogenic composition with subsequent booster administrations. In certain cases, a second dose of the immunogenic composition is administered anywhere from two weeks to one year, preferably from one to six months, after the initial administration. Additionally, a third dose may be administered after the second dose and from three months to two years, or even longer, preferably 4 to 6 months, or 6 months to one year after the initial administration. In certain cases, no booster immunization is required.

VIII. Determination of Therapeutic Efficacy

The invention encompasses methods for determining the efficacy of immunogenic compositions of the invention using any standard method known in the art or described herein. Assays for determining the efficacy of the immunogenic compositions of the invention may be in vitro based assays or in vivo based assays, including animal based assays. In some embodiments, the invention encompasses detecting and/or quantitating a humoral immune response against the antigenic or immunogenic agent of a composition of the invention in a sample, e.g., serum or mucosal wash, obtained from a subject who has been administered an immunogenic composition of the invention. Preferably, the humoral immune response of the immunogenic compositions of the invention are compared to a control sample obtained from the same subject prior to administration with the inventive formulation or after an individual has been administered a control formulation, e.g., a formulation which simply comprises of the antigenic or immunogenic agent.

In some embodiments, the invention encompasses detecting and/or quantitating a humoral immune response against the antigenic or immunogenic agent of the immunogenic composition of this invention in a sample, e.g., serum, obtained from a subject who has been administered an immunogenic composition of this invention. The humoral immune response of the immunogenic composition of this invention is compared to a control sample obtained from the same subject, who has been administered a control formulation, e.g., a formulation which simply comprises of the antigenic or immunogenic agent or a formulation which comprises of the antigenic or immunogenic agent and a ligand for TLR.

Assays for measuring humoral immune response are well known in the art, e.g., see, Coligan et al., (eds.), 1997, Current Protocols in Immunology, John Wiley and Sons, Inc., Section 2.1. A humoral immune response may be detected and/or quantitated using standard methods known in the art including, but not limited to, an ELISA assay. The humoral immune response may be measured by detecting and/or quantitating the relative amount of an antibody which specifically recognizes an antigenic or immunogenic agent in the sera of a subject who has been treated with an immunogenic composition of this invention relative to the amount of the antibody in an untreated subject. ELISA assays can be used to determine total antibody titers in a sample obtained from a subject treated with a composition of the invention. In other embodiments, ELISA assays may be used to determine the level of specific antibody isotypes and antibodies to neutralizing epitopes using methods known in the art.

ELISA based assays comprise preparing an antigen, coating the well of a 96 well microtiter plate with the antigen, adding test and control samples containing antigen specific antibody, adding a detector antibody specific to the antibody in test and control samples that is conjugated to an enzyme (e.g., horseradish peroxidase or alkaline phosphatase) and incubating for a period of time, and detecting the presence of the antigen with a color yielding substrate. One of skill in the art would be knowledgeable as to the parameters that can be modified to increase the signal detected as well as other variations of ELISAs known in the art. For further discussion regarding ELISAs see, e.g., Ausubel et al., eds, 1994, Current Protocols in Molecular Biology, Vol. 1, John Wiley & Sons, Inc., New York at 11.2.1.

The invention encompasses methods for determining the efficacy of the compositions of the invention by measuring cell-mediated immune response. Methods for measuring cell-mediated immune response are known to one skilled in the art and encompassed within the invention. In some embodiments, a T cell immune response may be measured for quantitating the immune response in a subject, for example by measuring cytokine production using common methods known to one skilled in the art including but not limited to ELISA from tissue culture supernatants, flow cytometry based intracellular cytokine staining of cells ex vivo or after an in vitro culture period, and cytokine bead array flow cytometry based assay. In yet other embodiments, the invention encompasses measuring T cell specific responses using common methods known in the art, including but not limited to chromium based release assay, flow cytometry based tetramer or dimer staining assay using known CTL epitopes.

IX. Prophylactic and Therapeutic Uses

The invention provides methods of treatment and prophylaxis which involve administering an immunogenic composition of the invention to a subject, preferably a mammal, and most preferably a human for treating, managing or ameliorating symptoms associated with a disease or disorder, especially an infectious disease or cancer. The subject is preferably a mammal such as a non-primate, e.g., cow, pig, horse, cat, dog, rat, mouse and a primate, e.g., a monkey such as a Cynomolgous monkey and a human. In a preferred embodiment, the subject is a human. Preferably, the immunogenic composition of the invention is a vaccine composition.

The invention encompasses a method for immunization and/or stimulating an immune response in a subject comprising delivering a single dose of a composition of the invention to a subject, preferably a human. In some embodiments, the invention encompasses one or more booster immunizations. The immunogenic composition of the invention is particularly effective in stimulating and/or up-regulating an antibody response to a level greater than that seen in immunogenic compositions lacking chemoattractant adjuvant. For example, an immunogenic composition of the invention may lead to an antibody response comprising generations of one or more antibody classes, such as IgM, IgG, and/or IgA. Most preferably, the immunogenic compositions of the invention including vaccine formulations stimulate a systemic immune response that protects the subject from at least one pathogen. The immunogenic compositions of the invention including vaccine compositions may provide systemic, local, or mucosal immunity or a combination thereof.

X. Target Diseases

The invention encompasses immunogenic compositions to treat and/or prevent an infectious disease in a subject preferably a human. Infectious diseases that can be treated or prevented by the methods of the present invention are caused by infectious agents including, but not limited to, viruses, bacteria, fungi protozoa, helminths, and parasites.

Examples of viruses that have been found in humans and can be treated by the immunogenic compositions and methods of the invention include, but are not limited to, Retroviridae (e.g., human immunodeficiency viruses, such as HIV-1 (also referred to as HTLV-III, LAV or HTLV-III/LAV, or HIV-III; and other isolates, such as HIV-LP); Picornaviridae (e.g., polio viruses, hepatitis A virus; enteroviruses, human Coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); avian flu H5N1 strain; Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (e.g., hemorrhagic fever viruses); Reoviridae (e.g., reoviruses, orbiviruses and rotaviruses); B imaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxyiridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted, e.g., Hepatitis C); Norwalk and related viruses, and astroviruses.

Retroviruses that results in infectious diseases in animals and humans and can be treated and/or prevented using the immunogenic compositions and methods of the invention include both simple retroviruses and complex retroviruses. The simple retroviruses include the subgroups of B-type retroviruses, C-type retroviruses and D-type retroviruses. An example of a B-type retrovirus is mouse mammary tumor virus (MMTV). The C-type retroviruses include subgroups C-type group A (including Rous sarcoma virus (RSV), avian leukemia virus (ALV), and avian myeloblastosis virus (AMV)) and C-type group B (including murine leukemia virus (MLV), feline leukemia virus (FeLV), murine sarcoma virus (MSV), gibbon ape leukemia virus (GALV), spleen necrosis virus (SNV), reticuloendotheliosis virus (RV) and simian sarcoma virus (SSV)). The D-type retroviruses include Mason-Pfizer monkey virus (MPMV) and simian retrovirus type 1 (SRV-1). The complex retroviruses include the subgroups of lentiviruses, T-cell leukemia viruses and the foamy viruses. Lentiviruses include HIV-1, but also include HIV-2, SIV, Visna virus, feline immunodeficiency virus (FIV), and equine infectious anemia virus (EIAV). The T-cell leukemia viruses include HTLV-1, HTLV-II, simian T-cell leukemia virus (STLV), and bovine leukemia virus (BLV). The foamy viruses include human foamy virus (HFV), simian foamy virus (SFV) and bovine foamy virus (BFV).

Examples of RNA viruses that are antigens in vertebrate animals include, but are not limited to, the following: members of the family Reoviridae, including the genus Orthoreovirus (multiple serotypes of both mammalian and avian retroviruses), the genus Orbivirus (Bluetongue virus, Eugenangee virus, Kemerovo virus, African horse sickness virus, and Colorado Tick Fever virus), the genus Rotavirus (human rotavirus, Nebraska calf diarrhea virus, murine rotavirus, simian rotavirus, bovine or ovine rotavirus, avian rotavirus); the family Picornaviridae, including the genus Enterovirus (poliovirus, Coxsackie virus A and B, enteric cytopathic human orphan (ECHO) viruses, hepatitis A virus, Simian enteroviruses, Murine encephalomyelitis (ME) viruses, Poliovirus muris, Bovine enteroviruses, Porcine enteroviruses, the genus Cardiovirus (Encephalomyocarditis virus (EMC), Mengovirus), the genus Rhinovirus (Human rhinoviruses including at least 113 subtypes; other rhinoviruses), the genus Apthovirus (Foot and Mouth disease (FMDV); the family Calciviridae, including Vesicular exanthema of swine virus, San Miguel sea lion virus, Feline picornavirus and Norwalk virus; the family Togaviridae, including the genus Alphavirus (Eastern equine encephalitis virus, Semliki forest virus, Sindbis virus, Chikungunya virus, O'Nyong-Nyong virus, Ross river virus, Venezuelan equine encephalitis virus, Western equine encephalitis virus), the genus Flavirus (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); forest virus, Sindbis virus, Chikungunya virus, O'Nyong-Nyong virus, Ross river virus, Venezuelan equine encephalitis virus, Western equine encephalitis virus), the genus Flavirus (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); the family Rhabdoviridae, including the genus Vesiculovirus (VSV), Chandipura virus, Flanders-Hart Park virus), the genus Lyssavirus (Rabies virus), fish Rhabdoviruses, and two probable Rhabdoviruses (Marburg virus and Ebola virus); the family Arenaviridae, including Lymphocytic choriomeningitis virus (LCM), Tacaribe virus complex, and Lassa virus; the family Coronoaviridae, including Infectious Bronchitis Virus (IBV), Mouse Hepatitis virus, Human enteric corona virus, and Feline infectious peritonitis (Feline coronavirus).

Illustrative DNA viruses that are antigens in vertebrate animals include, but are not limited to: the family Poxyiridae, including the genus Orthopoxvirus (Variola major, Variola minor, Monkey pox Vaccinia, Cowpox, Buffalopox, Rabbitpox, Ectromelia), the genus Leporipoxvirus (Myxoma, Fibroma), the genus Avipoxvirus (Fowlpox, other avian poxvirus), the genus Capripoxvirus (sheeppox, goatpox), the genus Suipoxvirus (Swinepox), the genus Parapoxvirus (contagious postular dermatitis virus, pseudocowpox, bovine papular stomatitis virus); the family Iridoviridae (African swine fever virus, Frog viruses 2 and 3, Lymphocystis virus of fish); the family Herpesviridae, including the alpha-Herpesviruses (Herpes Simplex Types 1 and 2, Varicella-Zoster, Equine abortion virus, Equine herpes virus 2 and 3, pseudorabies virus, infectious bovine keratoconjunctivitis virus, infectious bovine rhinotracheitis virus, feline rhinotracheitis virus, infectious laryngotracheitis virus) the Beta-herpesviruses (Human cytomegalovirus and cytomegaloviruses of swine, monkeys and rodents); the gamma-herpesviruses (Epstein-Barr virus (EBV), Marek's disease virus, Herpes saimiri, Herpesvirus ateles, Herpesvirus sylvilagus, guinea pig herpes virus, Lucke tumor virus); the family Adenoviridae, including the genus Mastadenovirus (Human subgroups A, B, C, D, E and ungrouped; simian adenoviruses (at least 23 serotypes), infectious canine hepatitis, and adenoviruses of cattle, pigs, sheep, frogs and many other species, the genus Aviadenovirus (Avian adenoviruses); and non-cultivatable adenoviruses; the family Papoviridae, including the genus Papillomavirus (Human papilloma viruses, bovine papilloma viruses, Shope rabbit papilloma virus, and various pathogenic papilloma viruses of other species), the genus Polyomavirus (polyomavirus, Simian vacuolating agent (SV-40), Rabbit vacuolating agent (RKV), K virus, BK virus, JC virus, and other primate polyoma viruses such as Lymphotrophic papilloma virus); the family Parvoviridae including the genus Adeno-associated viruses, the genus Parvovirus (Feline panleukopenia virus, bovine parvovirus, canine parvovirus, Aleutian mink disease virus, etc). Finally, DNA viruses may include viruses which do not fit into the above families such as Kuru and Creutzfeldt-Jacob disease viruses and chronic infectious neuropathic agents.

Bacterial infections or diseases that can be treated or prevented by the methods of the present invention are caused by bacteria including, but not limited to, bacteria that have an intracellular stage in its life cycle, such as mycobacteria (e.g., Mycobacteria tuberculosis, M. bovis, M. avium, M. leprae, or M. africanum), rickettsia, mycoplasma, chlamydia, and legionella. Other examples of bacterial infections contemplated include but are not limited to infections caused by Gram positive bacillus (e.g., Listeria, Bacillus such as Bacillus anthracis (anthrax antigen), Erysipelothrix species), Gram negative bacillus (e.g., Bartonella, Brucella, Campylobacter, Enterobacter, Escherichia, Francisella, Hemophilus, Klebsiella, Morganella, Proteus, Providencia, Pseudomonas, Salmonella, Serratia, Shigella, Vibrio, and Yersinia species), spirochete bacteria (e.g., Borrelia species including Borrelia burgdorferi that causes Lyme disease), anaerobic bacteria (e.g., Actinomyces and Clostridium species), Gram positive and negative coccal bacteria, Enterococcus species, Streptococcus species, Pneumococcus species, Staphylococcus species, Neisseria species. Specific examples of infectious bacteria include but are not limited to: Helicobacter pyloris, Borelia burgdorferi, Legionella pneumophilia, Mycobacteria tuberculosis, M. avium, M. intracellulare, M. kansaii, M. gordonae, Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus viridans, Streptococcus faecalis, Streptococcus bovis, Streptococcus pneumoniae, Haemophilus influenzae, Bacillus antracis, corynebacterium diphtheriae, Erysipelothrix rhusiopathiae, Clostridium perfringers, Clostridium tetani, Enterobacter aerogenes, Klebsiella pneumoniae, Pasturella multocida, Fusobacterium nucleatum, Streptobacillus moniliformis, Treponema pallidium, Treponema pertenue, Leptospira, Rickettsia, and Actinomyces israelli.

Fungal diseases that can be treated or prevented by the methods of the present invention include but not limited to aspergilliosis, crytococcosis, sporotrichosis, coccidioidomycosis, paracoccidioidomycosis, histoplasmosis, blastomycosis, zygomycosis, and candidiasis.

Parasitic diseases that can be treated or prevented by the methods of the present invention including, but not limited to, amebiasis, malaria, leishmania, coccidia, giardiasis, cryptosporidiosis, toxoplasmosis, and trypanosomiasis. Also encompassed are infections by various worms, such as but not limited to ascariasis, ancylostomiasis, trichuriasis, strongyloidiasis, toxoccariasis, trichinosis, onchocerciasis. filaria, and dirofilariasis. Also encompassed are infections by various flukes, such as but not limited to schistosomiasis, paragonimiasis, and clonorchiasis. Parasites that cause these diseases can be classified based on whether they are intracellular or extracellular. An “intracellular parasite” as used herein is a parasite whose entire life cycle is intracellular. Examples of human intracellular parasites include Leishmania spp., Plasmodium spp., Trypanosoma cruzi, Toxoplasma gondii, Babesia spp., and Trichinella spiralis. An “extracellular parasite” as used herein is a parasite whose entire life cycle is extracellular. Extracellular parasites capable of infecting humans include Entamoeba histolytica, Giardia lamblia, Enterocytozoon bieneusi, Naegleria and Acanthamoeba as well as most helminths. Yet another class of parasites is defined as being mainly extracellular but with an obligate intracellular existence at a critical stage in their life cycles. Such parasites are referred to herein as “obligate intracellular parasites”. These parasites may exist most of their lives or only a small portion of their lives in an extracellular environment, but they all have at least one obligate intracellular stage in their life cycles. This latter category of parasites includes Trypanosoma rhodesiense and Trypanosoma gambiense, Isospora spp., Cryptosporidium spp, Eimeria spp., Neospora spp., Sarcocystis spp., and Schistosoma spp.

The invention also encompasses vaccine compositions to treat and/or prevent cancers, including, but not limited to, neoplasms, tumors, metastases, or any disease or disorder characterized by uncontrolled cell growth. For example, but not by way of limitation, cancers and tumors associated with the cancer and tumor antigens listed supra may be treated and/or prevented using the vaccine compositions of the invention.

XI. Screening Methods to Identify Chemoattractant Adjuvants

The invention further encompasses methods of identifying a chemoattractant compound that enhances immunogenicity of an immunogenic or antigenic agent when delivered to a subject.

In some embodiments, methods of identifying a chemoattractant compound that enhances immunogenicity of an immunogenic or antigenic agent when delivered to a subject comprise stability measurements of such compounds. In a specific embodiment, candidate chemoattractant compounds or agents are combined with an immunogenic or antigenic agent and a ligand for TRL at a variety of ratios to prepare an immunogenic composition and the resulting composition is monitored for signs of instability relative to the immunogenic or antigenic agent alone or in combination with a ligand for TLR in real time and accelerated studies. Stability of the compositions may be assessed using methods known to one skilled in the art and disclosed herein.

In other embodiments, methods of identifying a chemoattractant compound that enhances immunogenicity of an immunogenic or antigenic agent when delivered to a subject comprises delivering the candidate chemoattractant compound to a subject. In some embodiments, the candidate compounds are delivered at a variety of concentrations and monitored for any indications of toxicity using standard methods known to one skilled in the art. Concentrations of candidate compound that do not contribute to degradation and/or toxicity of the immunogenic or antigenic agent in animal pre-trials are then combined with the immunogenic or antigenic agent and ligand for TLR and evaluated for adjuvant properties in a subject using methods disclosed and exemplified herein. Adjuvant properties may be assayed using any of the humoral or cell-based assays disclosed herein or any other method known to one skilled in the art.

In other embodiments, in order to identify such chemoattractant compounds an immunogenic or antigenic agent and a ligand for TLR are administered together with a candidate chemoattractant compound to a subject; the immune response resulting from the administration is determined; the same immunogenic or antigenic agent and ligand for TLR are administered without the candidate chemoattractant compound to a second subject, preferably of the same species; the immune response resulting from the second administration is determined using methods known to one skilled in the art; and the immune responses from the first and second administrations are compared. If the immune response from the first administration is greater than the first administration, the chemoattractant compound is characterized as a lead compound, wherein it has adjuvant activity.

The immune response in the subject resulting from the administration of an immunogenic or antigenic agent and ligand for TLR, with or without the candidate compound, may be determined using any methods known in the art or the methods disclosed herein. The assay for determining the immune response may be in vitro based assays or in vivo based assays, including animal based assays. The invention encompasses measuring humoral based and cell based immune responses using standard methods known to one skilled in the art and described above. Preferably, the screening assays of the invention are done in a high through put manner.

In a specific embodiment, a method for identifying a compound that enhances immunogenicity of an immunogenic or antigenic agent comprises: (a) delivering an immunogenic composition into a first subject, wherein the immunogenic composition comprises the immunogenic or antigenic agent, a ligand for TLR, and the chemoattractant compound; (b) measuring antibody response in a sample obtained from the first subject's serum; (c) delivering and immunogenic composition into a second subject, wherein the immunogenic composition comprises the immunogenic or antigenic agent, a ligand for TLR, without the chemoattractant compound, and wherein the first and the second subjects are same species; (d) measuring antibody response in a sample obtained from the second subject's serum; and (e) determining whether the response obtained from the first subject is greater than the response obtained from the second subject. If the response in the sample obtained from the first subject is greater than the second subject, characterizing the compound as an adjuvant that may be used in the compositions of the invention. Chemoattractant compounds identified by the screening methods of the invention may be used to elicit an enhanced immune response to an antigenic or immunogenic agent when co-administered with the antigenic or immunogenic agent and a ligand for TLR into a subject. Specifically, these compounds can be used in vaccine compositions.

The compounds used in the assays described herein are members of chemoattractant family of compounds described above.

Kits

The invention further comprises kits comprising an immunogenic composition of the invention as described herein. In some embodiments, the kit may further include a standard administration device. In some embodiments, the invention also provides a pharmaceutical pack or kit comprising an immunogenic composition of the invention. In a specific embodiment the invention provides a kit comprising, one or more containers filled with one or more of the components of the immunogenic compositions of the invention, e.g., an antigenic or immunogenic agent, a ligand for TLR, and a chemoattractant. In yet another embodiment the pre-filled container further comprises a delivery device. In another specific embodiment, the kit comprises two containers, one containing an antigenic or immunogenic agent, and the other containing the ligand for TLR and the chemoattactant. Alternatively, the kit comprises three containers, one containing an antigenic or immunogenic agent, one containing the ligand for TLR, and one containing a chemoattactant. Associated with such container(s) may be a notice in the form prescribed by a governmental agency regulating the manufacture, use or sale of pharmaceuticals or biological products, which notice reflects approval by the agency of manufacture, use or sale for human administration.

Aspects of this invention are illustrated by the following non-limiting examples.

### EXAMPLES

Methods

Animals: Female BALB/C mice 8-10 weeks of age were purchased from Charles River Laboratories (Hollister, Calif.). Animals were housed in microisolator caging and provided food and water ad libitum. Mice were allowed to acclimate for 7 days prior to experimentation. All procedures for use and care of mice were approved by the institutional animal care and use committee.

Immunizations and Sampling: Mice were intranasally immunized with 10 μgs of recombinant anthrax protective antigen (rPA) (List Biological Laboratories, Campbell, Calif.) in a total volume of 30 μLs (15 μLs/nostril) under light isoflurane anesthesia. For adjuvant groups either chemokine or CpG-ODN 1668 (5′-TCCATGACGTTCCTGATGCT-3′; SEQ ID NO: 59) alone or in combination were mixed in saline as indicated in the results sections. The volume for these immunizations was kept consistent with that of the control group (rPA without adjuvant). Animals were harvested on day 21 for antigen specific endpoint determinations by CO₂ exposure.

Individual chemokine concentrations were as follows: SHAAGtide (MLWRRKIGPQMTLSHAAG-CONH₂; SEQ ID NO: 1) (100 μg per animal per immunization), W-tide (WKYMV-(d-Met)-CONH₂; SEQ ID NO: 27) (30 μg per animal per immunization), mJE (SEQ ID NO: 60) (10 μg per animal per immunization) and mC5a (SEQ ID NO: 62) (10 μg per animal per immunization). A sequence of full length mJE is shown in SEQ ID NO: 61.

Lavage was carried out on various mucosal surfaces as indicated. Nasal lavage was conducted by removing the lower jaw and inserting a catheter above the hard palate and flushing with 200 mLs of saline. Bronchalveolar lavage (BAL) was conducted by inserting catheter tubing into the trachea, 700 mLs of saline was used to conduct the lavage. Vaginal lavages were conducted by flushing the area with 50mls of saline.

Antibody Specific ELISA: Antigen specific antibodies were determined from samples taken at day 21 following the immunizations as follows. An ELISA was used to determine the levels of Ag-specific Abs in the serum, nasal, vaginal and bronchalveolar lavage (BAL) samples. ELISA plates were coated with rPA at 1 μg/mL overnight at 4° C. Plates were washed (Phosphate Buffered Saline (PBS) and 0.1% Tween 20) three times prior to blocking with 5% Fetal Bovine Serum (FBS) in PBS for 1 hour. Samples were diluted in 5% FBS/PBS as appropriate and incubated for 2 hours. Ag-specific Ab concentration was determined from a standard curve of anti-PA IgG2b (Chemicon, Temecula, Calif.). Plates were washed (PBS 0.1% Tween 20) three times prior to addition of secondary antibodies (1 :1500 dilution) targeted against IgG1, IgG2a, IgG2b and IgA (a specific secreted subclass of antibodies important in mucosal immunity) as appropriate for 1 hour. Plates were read at 540 nM using an automated 96 well plate reader as per manufacturer's instructions.

### Example 1

### SHAAGtide-Induced Enhancement of Immune Response to an Antigen

Mice were immunized with (1) rPA antigen alone, (2) rPA and CpG, (3) rPA and SHAAGtide, and (4) rPA and combination of CpG and SHAAGtide, as specified above.

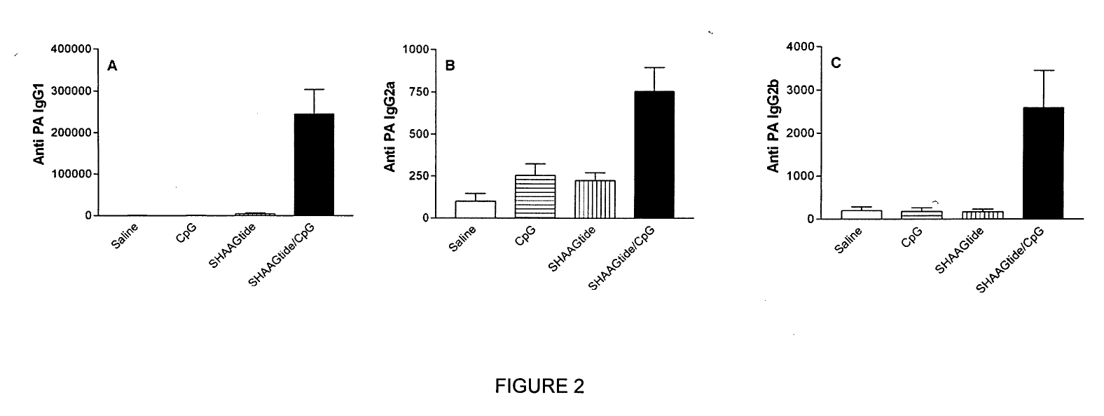

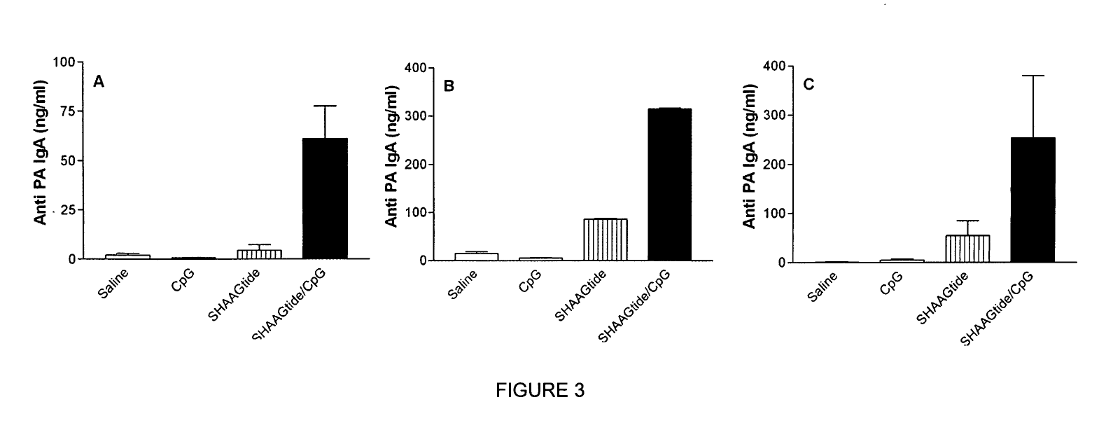

At day 21 following immunizations, levels of anti-PA IgG1, anti-PA IgG2a, and IgG2b antibodies were measured in serum samples (FIGS. 2A-C), as described above. Also levels of IgA antibodies in mucosal samples (FIGS. 3A-C) were measured as described above.

As illustrated in FIGS. 2A-C, both SHAAGtide and CpG when used alone did not induce significant levels of antigen specific IgG antibodies. When SHAAGtide and CpG were used in combination (black bar in FIGS. 2A-C), a significant increase in serum levels of antigen specific IgG1 (A), IgG2a (B), and IgG2b (C) were observed.

As illustrated in FIGS. 3A-C, both SHAAGtide and CpG when used alone did not induce significant levels of antigen specific IgA antibodies. When SHAAGtide and CpG were used in combination (black bar in FIGS. 3A-C), a significant increase in levels of antigen specific IgA antibodies in nasal (A), the lung (B), and vaginal (C) mucosa was observed.

### Example 2

### W-tide-Induced Enhancement of Immune Response to an Antigen

Mice were immunized with (1) rPA antigen alone, (2) rPA and CpG, (3) rPA and W-tide, and (4) rPA and combination of CpG and W-tide, as specified above.

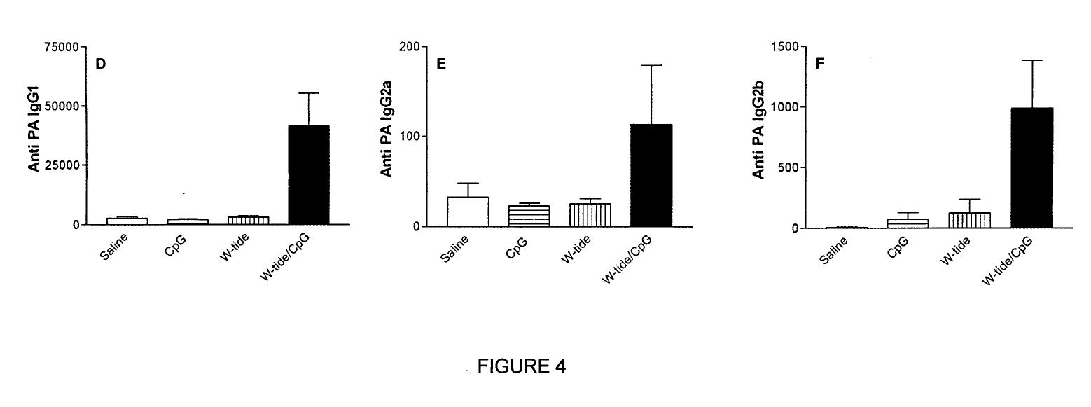

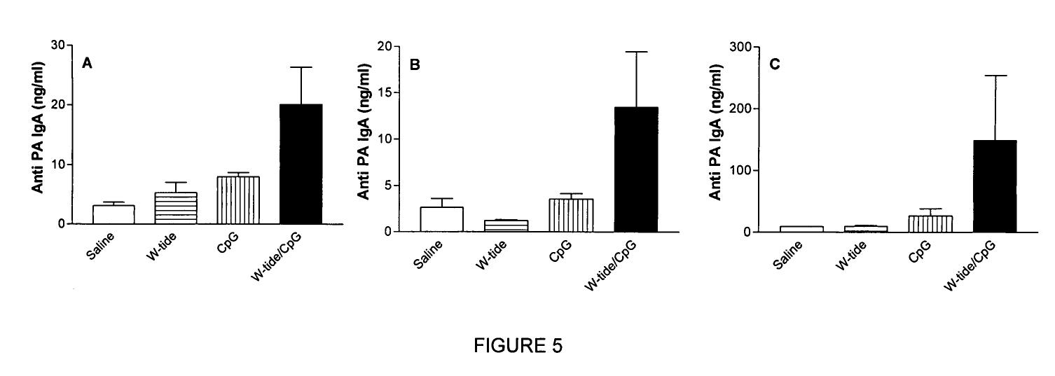

At day 21 following immunizations, levels of anti-PA IgG1, anti-PA IgG2a, and IgG2b antibodies were measured in serum samples (FIGS. 4A-C), as described above. Also levels of IgA antibodies in mucosal samples (FIGS. 5A-C) were measured as described above.

As illustrated in FIGS. 4A-C, both W-tide and CpG when used alone did not induce significant levels of antigen specific IgG antibodies. When W-tide and CpG were used in combination (black bar in FIGS. 4A-C), a significant increase in serum levels of antigen specific IgG1 (A), IgG2a (B), and IgG2b (C) were observed.

As illustrated in FIGS. 5A-C, both W-tide and CpG when used alone did not induce significant levels of antigen specific IgA antibodies. When W-tide and CpG were used in combination (black bar in FIGS. 5A-C), a significant increase in levels of antigen specific IgA antibodies in nasal (A), the lung (B), and vaginal (C) mucosa was observed.

### Example 3

### mJE-Induced Enhancement of Immune Response to an Antigen

Mice were immunized with (1) rPA antigen alone, (2) rPA and CpG, (3) rPA and mJE, and (4) rPA and combination of CpG and mJE, as specified above.

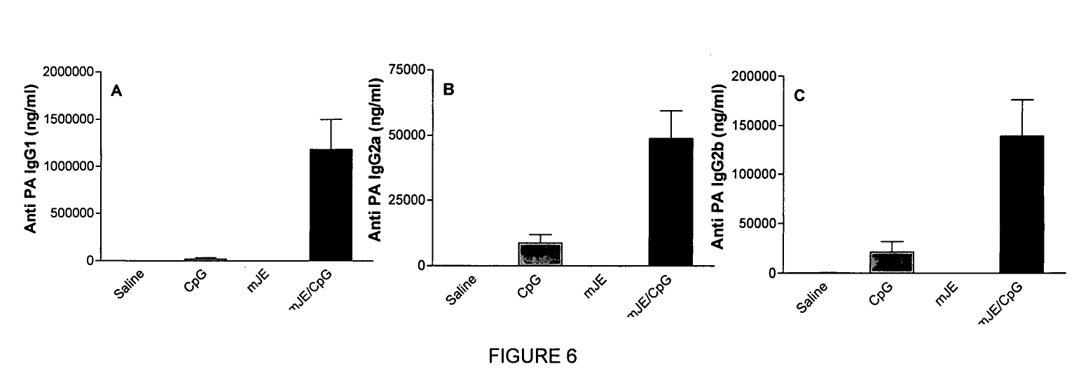

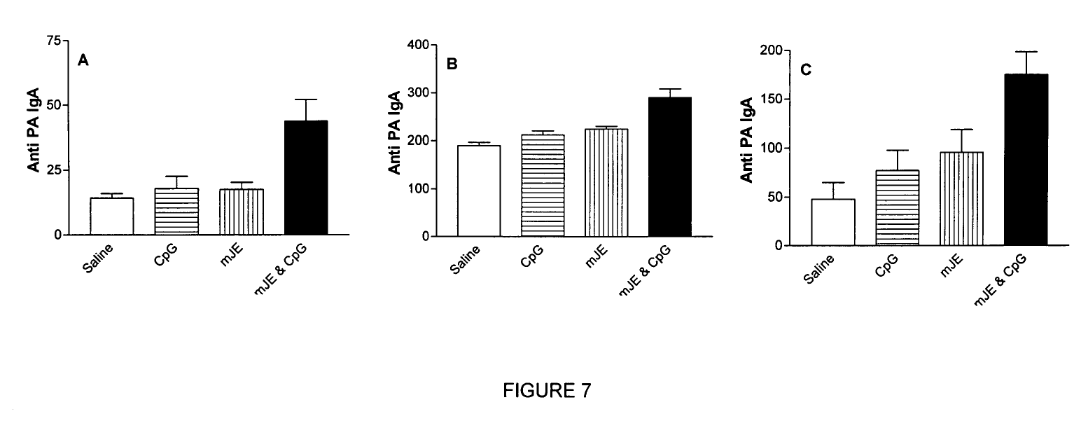

At day 21 following immunizations, levels of anti-PA IgG1, anti-PA IgG2a, and IgG2b antibodies were measured in serum samples (FIGS. 6A-C), as described above. Also levels of IgA antibodies in mucosal samples (FIGS. 7A-C) were measured as described above.

As illustrated in FIGS. 6A-C, both mJE and CpG when used alone did not induce significant levels of antigen specific IgG antibodies. When mJE and CpG were used in combination (black bar in FIGS. 6A-C), a significant increase in serum levels of antigen specific IgG1 (A), IgG2a (B), and IgG2b (C) were observed.

As illustrated in FIGS. 7A-C, both mJE and CpG when used alone did not induce significant levels of antigen specific IgA antibodies. When mJE and CpG were used in combination (black bar in FIGS. 7A-C), a significant increase in levels of antigen specific IgA antibodies in nasal (7A), the lung (7B), and vaginal (7C) mucosa was observed.

### Example 4

### Chemokine (mC5a)-Induced Enhancement of Immune Response to an Antigen

Mice were immunized with (1) rPA antigen alone, (2) rPA and CpG, (3) rPA and mC5a, and (4) rPA and combination of CpG and mC5a, as specified above.

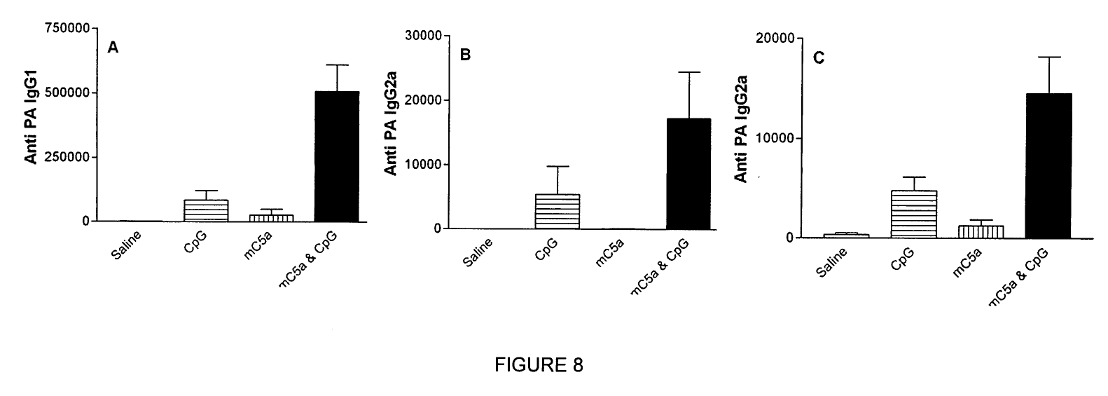

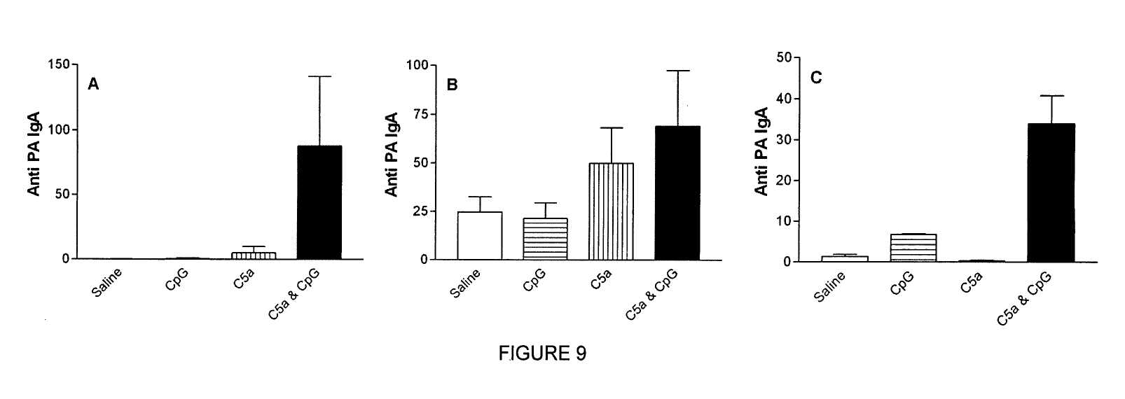

At day 21 following immunizations, levels of anti-PA IgG1, anti-PA IgG2a, and IgG2b antibodies were measured in serum samples (FIGS. 8A-C), as described above. Also levels of IgA antibodies in mucosal samples (FIGS. 9A-C) were measured as described above.

As illustrated in FIGS. 8A-C, both mC5a and CpG when used alone did not induce significant levels of antigen specific IgG antibodies. When mC5a and CpG were used in combination (black bar in FIGS. 8A-C), a significant increase in serum levels of antigen specific IgG1 (8A), IgG2a (8B), and IgG2b (8C) were observed.

As illustrated in FIGS. 9A-C, both mC5a and CpG when used alone did not induce significant levels of antigen specific IgA antibodies. When mC5a and CpG were used in combination (black bar in FIGS. 9A-C), a significant increase in levels of antigen specific IgA antibodies in nasal (9A), the lung (9B), and vaginal (9C) mucosa was observed.

### Example 5

### Chemoattractant-Induced Enhancement of Immune Response to an Antigen

Mice were immunized with (1) rPA antigen alone, (2) rPA and SHAAGtide, (3) rPA and W-tide, (4) rPA and CpG, (5) rPA and combination of CpG and SHAAGtide, and (6) rPA and combination of CpG and W-tide as specified above.

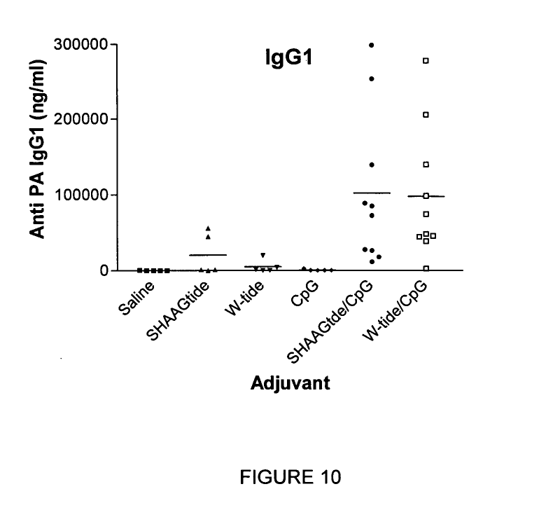

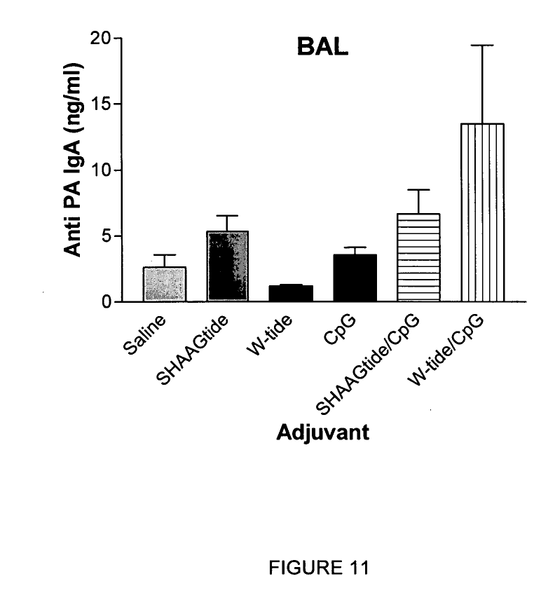

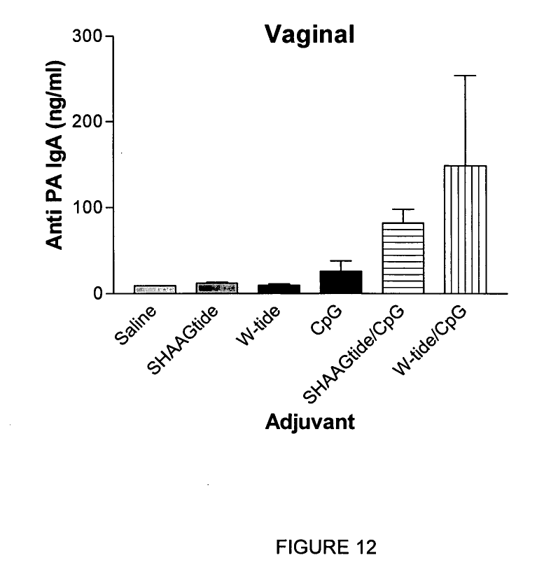

At day 21 following immunizations, levels of anti-PA IgG1 antibodies were measured in serum samples (FIG. 10), as described above. Also levels of IgA antibodies in mucosal samples (FIGS. 11 and 12) were measured as described above.

As illustrated in FIG. 11, all three, SHAAGtide, W-tide, and CpG when used alone did not induce significant levels of antigen specific IgG antibodies. When SHAAGtide and CpG were used in combination, a significant increase in serum levels of antigen specific IgG1 antibodies was observed. Also, when W-tide and CpG were used in combination, a significant increase in serum levels of antigen specific IgG1 antibodies was observed.

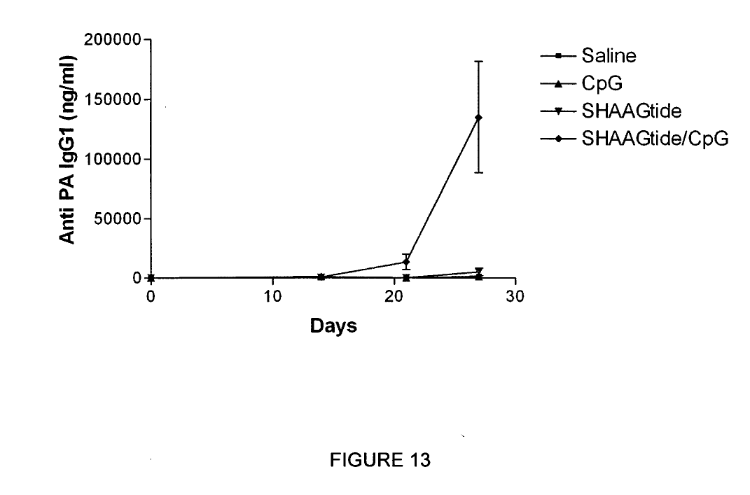

As illustrated in FIGS. 12 and 13, all three, SHAAGtide, W-tide, and CpG when used alone did not induce significant levels of antigen specific IgA antibodies. When SHAAGtide and CpG were used in combination, a significant increase in serum levels of antigen specific IgA antibodies in the lung (FIG. 12) and vaginal (FIG. 13) mucosa was observed. Also, when W-tide and CpG were used in combination, a significant increase in serum levels of antigen specific IgA antibodies in the lung (FIG. 12) and vaginal (FIG. 13) mucosa was observed. In general, the W-tide and CpG combination resulted in a greater immune response than one resulting from the SHAAGtide and CpG combination.

### Example 6

### SHAAGtide-Induced Enhancement of Immune Response to an Antigen

Mice were immunized with (1) rPA antigen alone, (2) rPA and CpG, (3) rPA and SHAAGtide, and (4) rPA and combination of CpG and SHAAGtide, as specified above by intranasal inoculation.

Levels of anti-PA IgG1 antibodies were measured in serum samples (FIG. 13) at various timed following immunization, as described above.

Specifically, FIG. 13 shows levels of serum IgG specific for PA antigen when the PA antigen is combined with Saline (negative control), SHAAGtide, CpG, or a combination of SHAAGtide and CpG. The SHAAG/CpG response was significantly higher as compared to other treatment groups.

### Example 7

### W-tide-Induced Enhancement of Immune Response to an Antigen

Mice were immunized with (1) rPA antigen alone, (2) rPA and CpG, (3) rPA and W-tide, and (4) rPA and combination of CpG and W-tide, as specified above by intra-nasal inoculation.

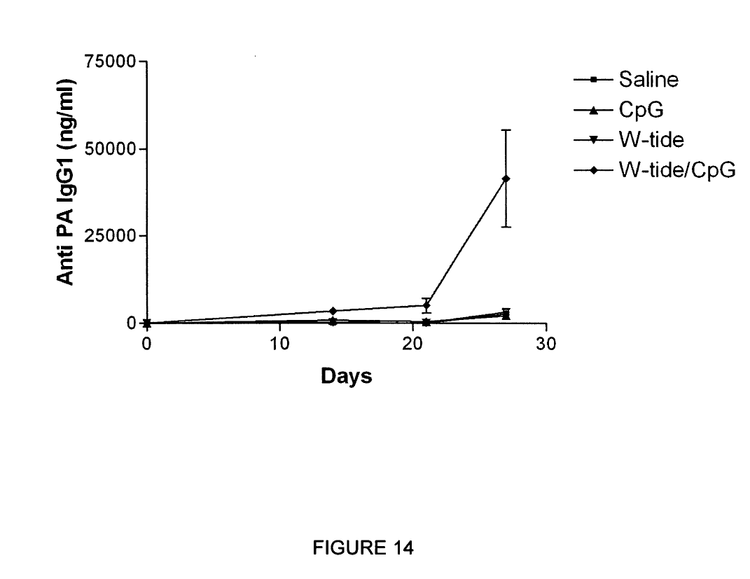

Levels of anti-PA IgG1 antibodies were measured in serum samples (FIG. 14) at various timed following immunization, as described above.

FIG. 14 shows levels of serum IgG specific for PA antigen when the PA antigen is combined with Saline (negative control), W-tide, CpG, or a combination of W-tide and CpG. The W-tide/CpG response was significantly higher as compared to other treatment groups.

### Example 8

### SHAAGtide-Induced Enhancement of Mucosal Immunity

Mice were immunized with (1) rPA antigen alone, (2) rPA and CpG, (3) rPA and SHAAGtide, and (4) rPA and combination of CpG and SHAAGtide, as specified above by intra-nasal inoculation.

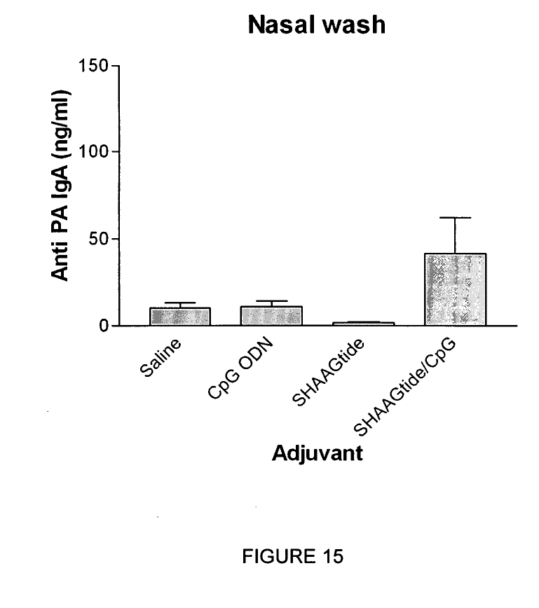

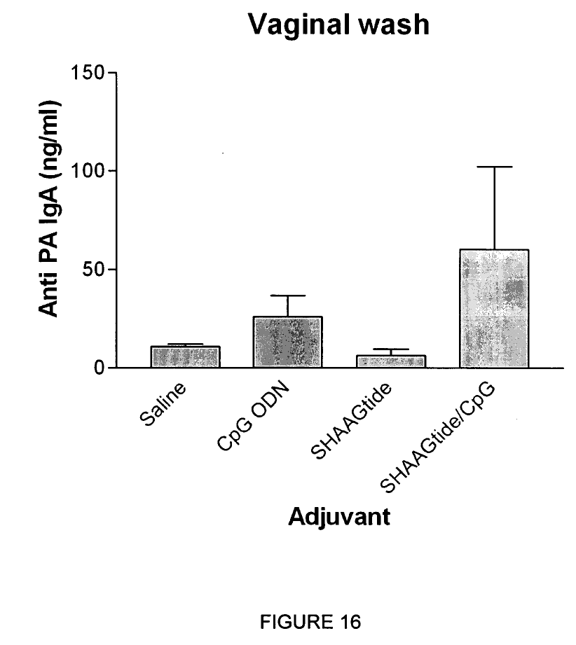

Levels of anti-PA IgA antibodies were measured in nasal (FIG. 15) and vaginal (FIG. 16) wash 27 days following immunization.

Specifically, FIG. 15 shows the titers of PA antigen-specific IgA recovered from the site of inoculation by nasal wash. The combination of adjuvants induced significantly higher production of PA antigen specific antibodies as compared to either treatment alone. FIG. 16 shows levels of PA antigen-specific IgA recovered at a site distal to that of inoculation, e.g., from a vaginal wash. Here again, the combination of adjuvants induced significantly higher production of PA antigen-specific antibodies as compared to other treatments.

It is therefore intended that the foregoing detailed description be regarded as illustrative rather than limiting, and that it be understood that it is the following claims, including all equivalents, that are intended to define the spirit and scope of this invention.

1. An immunogenic composition, comprising:
an antigen or immunogen;
at least one chemoattractant; and
a ligand for a Toll-like receptor (TLR).

2. The immunogenic composition of claim 1, wherein the ligand for TLR is a TLR9 ligand.

3. The immunogenic composition of claim 2, wherein the TLR9 ligand is CpG oligonucleotide.

4. The immunogenic composition of claim 1, wherein the chemoattractant is a chemokine or a w-peptide.

5. The immunogenic composition of claim 4, wherein the chemokine is selected from the group consisting of IL-8, GCP-2, Gro α, Gro β, Gro γ, ENA-78, PBP, MIG, IP-10, I-TAC, SDF-1α (PBSF), BLC (BCA-1), MIP-1α, MIP-1β, RANTES, HCC-1, -2, -3, and 4, MCP-1, -2, -3, and -4, eotaxin-1, eotaxin-2, TARC, MDC, MIP-3α (LARC), MIP-3β (ELC), 6Ckine (LC), I309, TECK, lymphotactin, fractalkine (neurotactin), TCA-4, Exodus-2, Exodus-3, and CKβ-11.

6. The immunogenic composition of claim 4, wherein the ligand for TLR is a TLR9 ligand.

7. The immunogenic composition of claim 1, wherein the chemoattractant is a SHAAGtide polypeptide having at least 80% sequence identity to SEQ ID NO: 1.

8. The immunogenic composition of claim 7, wherein the ligand for TLR is a TLR9 ligand.

9. The immunogenic composition of claim 1, wherein the chemoattractant is a SHAAGtide polypeptide selected from the group consisting of SEQ ID NOS: 1, 2, 3, 4, 5, 6, 7, 8, 9, 10, and 11.

10. The immunogenic composition of claim 4, wherein the w-peptide is selected from the group consisting of SEQ ID NOS: 31, 32, 33, 34, 35, 36, 37, 38, 39, 40, 41, 42, 43, 44, 45, 46, 47, 48, 49, 50, 51, 52, 53, 54, 55, 56, 57, and 58.

11. The immunogenic composition of claim 1, wherein the immunogenic composition is a vaccine composition.

12. The immunogenic composition of claim 1, wherein the antigen is a viral protein, polypeptide, or a fragment thereof; a cancer or tumor antigen; infectious disease agent; a bacterial agent; a parasite agent; or a fungal agent.

13. The immunogenic composition of claim 1, wherein the composition induces mucosal immunity in a subject.

14. The immunogenic composition of claim 13, wherein the subject is a mammal.

15. The immunogenic composition of claim 13, wherein the subject is a human.

16. The immunogenic composition of claim 1, wherein the composition enhances an antibody response in a subject.

17. The immunogenic composition of claim 1, wherein the composition is for treatment of an infectious disease.

18. The immunogenic composition of claim 1, wherein the composition is for treatment of a cancer.

19. A method of enhancing immunity, comprising delivering to a subject an effective amount of a composition comprising an antigen, at least one chemoattractant, and a ligand for a Toll Like Receptor (TLR).

20. The method of claim 19, wherein the ligand for TLR is a TLR9 ligand.

21. The method of claim 19, wherein the method enhances mucosal immunity.

22. The method of claim 19, further comprising a step of measuring a level of immune response to the immunogenic composition, wherein the immune response is directed to the antigen or immunogen.

23. The method of claim 22, wherein the step of measuring comprises determining humoral response.

24. The method of claim 22, wherein the step of measuring comprises determining cell-mediated immune response.

25. A method for identifying an adjuvant compound that enhances immunity to an antigen or immunogen, comprising
delivering an immunogenic composition comprising the antigen or immunogen, at least one chemoattractant and a ligand for a Toll-like receptor to a subject; and
measuring a level of immune response to the immunogenic composition, wherein the immune response is directed to the antigen or immunogen.

26. A method of inducing a mucosal immunity, comprising applying to a mucosal membrane in a subject an effective amount of a composition comprising an antigen or immunogen, at least one chemoattractant, and a CpG.

27. The method of claim 26, wherein the mucosal membrane is vaginal.

28. The method of claim 26, wherein the mucosal membrane is nasal.
